(19) United States
(12) Patent Application Publication
(10) Pub. No.: US 2024/0118268 A1
(43) Pub. Date: Apr. 11, 2024

(54) BIOMARKERS ON CELLULAR ENDOCRINE MODELS FOR ENDOCRINE DISRUPTION ASSESSMENT

(71) Applicants: CENTRE NATIONAL DE LA RECHERCHE SCIENTIFIQUE, Paris (FR); UNIVERSITÉ DE PARIS CITÉ, Paris (FR); YSLAB, Quimper (FR)

(72) Inventors: Patrice RAT, Paris (FR); Elodie OLIVIER, Versailles (FR); Melody DUTOT, Saint Maur des Fossés (FR); Anaïs WAKX, La Varenne Saint Hilaire (FR)

(21) Appl. No.: 18/257,244

(22) PCT Filed: Dec. 17, 2021
(86) PCT No.: PCT/EP2021/086657

§ 371 Date: Jun. 13, 2023

(30) Foreign Application Priority Data
Dec. 18, 2020 (EP) 20306607.1

(51) Int. Cl. G01N 33/50; C12N 5/073; G01N 33/74

(52) U.S. Cl. CPC G01N 33/5044; C12N 5/0605; G01N 33/743; G01N 2333/705; G01N 2333/90245

(57) ABSTRACT

The invention relates to a cell culture comprising a placental cell and a culture medium consisting of minimal essential nutriments and a low amount of serum. The invention also relates to a method using the cell culture for identifying endocrine disruptor.

The invention relates to endocrine cells, and their use in particular in the field of toxicology and endocrine disruptors assessment.

World Health Organization defines an Endocrine Disrupting Chemical (EDC) as an exogenous substance or mixture that alters function(s) of the endocrine system and consequently causes adverse health effects in an intact organism, or its progeny, or (sub)populations (WHO, 2002). EDCs and potential EDCs are mostly man-made and found in various materials such as plastics, metals, additives or contaminants in food and personal care products, so humans are continuously exposed to EDCs.

According to the European Union definition, endocrine-disrupting chemical substances (“endocrine disruptors”) are substances that alter the functions of the hormonal system and consequently cause adverse effects.

EDCs are found in several human tissues including maternal tissues during pregnancy: bisphenol A, triclosan, phthalates and parabens have been identified in pregnant women urines and phthalates in cord blood samples.

The problem is that exposure to EDCs during pregnancy can lead to many adverse pregnancy outcomes that are harmful for both the mother and the baby.

Phthalates, mainly dibutyl phthalate, can adversely affect fetal growth by gestational age reduction and preterm delivery, alkylphenols are associated with spontaneous abortion, parabens can influence birth outcomes and preterm birth, triclosan can disrupt gestational age and phthalates and bisphenols potentially disturb placental growth and function, which can lead to preeclampsia. Preeclampsia is a multisystem pregnancy-specific disorder and constitutes a major source of morbidity and mortality worldwide. Preeclampsia, but also miscarriage, is associated to placental dysfunctions.

Most of the studies carried out on placenta to study risks for the pregnancy consider it as a barrier between mother and fetus but not as an organ required for fetal development. It is not a true barrier since any chemical with a molecular weight <1000 daltons readily crosses the placenta from mother to fetus, and most of the pesticides, metals and EDCs have a molecular weight <1000. This potential transfer from mother to fetus was confirmed by studies that identified parabens, alkylphenols, bisphenols and 3-benzylidene camphor in human placentas, where they can concentrate.

Cellular Mechanisms of EDCs Leading to Pregnancy Disorders Remain Unclear

According to the WHO and European Union definition, if a modification of hormonal production is not formally linked to an adverse effect, the chemicals substances will not be considered as endocrine disruptors per se. It is therefore essential to provide a test that uniquely identify these compounds.

Some methods have been described in the art for identifying endocrine disruptors. For instance WO2007113204 discloses a chip array allowing to identify such kind of compounds. WO2011032284 discloses a cellular model comprising a steroid biosynthesis knock down nucleic acid allowing to identify endocrine disruptor.

However, these methods are not really specific, and do not correctly assess the link between the modulation of hormone secretion and the consequence in human health.

It is known from the art that compounds can induce expression of P2X7. This is the case of precedent article of inventors: Anais Waks et al.—Toxycology in vitro—2016—pp 76-85 which evaluate the metabolisation of benzo(A)pyrene. However, this compound has never been classified as a true endocrine disruptor. In this document, the authors have cultured endocrine cells in a medium containing 10% serum, and saw that, after treatment and deprivation of serum, no apoptosis is observed. This document never intended nor established that benzo(A)pyrene is an endocrine disruptor, nor that P2X7 is associated with endocrine disruptors.

Therefore, there is a need to provide a new efficient method that can identify the endocrine disruptors in an unambiguous manner.

One aim of the invention is to overcome the lack of the art.

Another aim of the invention is to provide a new efficient model for identifying the endocrine disruptors.

Still another aim of the invention is to provide a method that unambiguously identify if a compound having an effect on hormone secretion is an endocrine disruptor.

Thus, the invention relates to a cell culture comprising:
an endocrine cell, preferably a placental cell; and
a culture medium consisting of minimal essential nutriments and serum,
wherein said serum represents from 1.5 to 3.5% weight compared to the total weight of the culture medium.

The invention is based on the unexpected observation made by the inventors that endocrine cells that are cultured in a specific culture medium comprising minimum essential nutriments, and a determined amount of serum, are viable and able to proliferate, but become more sensitive to chemicals, and therefore constitute an ideal model for detecting and classifying endocrine disruptors.

The endocrine cells belonging to the above-described culture medium are cells able to produce and release hormones. These cells can be advantageously testis endocrine cells, ovarian endocrine cells, and placental endocrine cells, or any cells that are able to secrete at least one hormone.

Cell culture media generally comprise an appropriate source of energy and compounds which regulate the cell cycle. A typical culture medium is composed of a complement of amino acids, vitamins, inorganic salts, glucose. In addition to nutrients, the medium also helps maintain pH and osmolality.

The serum is provided as a source of growth factors, hormones, and attachment factors. this can be serum from calf, bovine or an artificially reconstituted serum comprising factors allowing in particular homeostasis, cell survival, proliferation . . . . The serum is preferably “decomplemented”, the components of the complement are inactivated by heat in order to induce protein precipitation/coagulation, according to well-known technics in the art.

In the invention, the serum represents “from 1.5 to 3.5%” compared to the total weight of the culture medium, which means that serum represents 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4 or 3.5% by weight compared to the total weight of the culture medium.

It is also possible to express the amount of serum in % v/v compared to the total volume of the cell culture, i.e. compared to the total volume of the medium comprising minimal essential nutriments.

Minimal essential nutriments are constituted by essential amino acids, vitamins, oligo elements etc. For instance, minimal essential nutriments according to the invention can be: Glycine, L-Alanine, L-Arginine hydrochloride, L-Asparagine-H2O, L-Aspartic acid, L-Cysteine hydrochloride-H2O, L-Cystine 2HCl, L-Glutamic Acid, L-Glutamine, L-Histidine, L-Histidine hydrochloride-H2O, L-Isoleucine, L-Leucine, L-Lysine hydrochloride, L-Methionine, L-Phenylalanine, L-Proline, L-Serine, L-Threonine, L-Tryptophan, L-Tyrosine disodium salt dihydrate, L-Valine, Ascorbic acid, Biotin, Choline chloride, D-Calcium pantothenate, Folic Acid, Niacinamide, Pyridoxal hydrochloride, Riboflavin, Thiamine hydrochloride, Vitamin B12, i-Inositol, Calcium Chloride (CaCl2) (anhyd.), Magnesium Sulfate (MgSO4) (anhyd.), Potassium Chloride (KCl), Sodium Bicarbonate (NaHCO₃), Sodium Chloride (NaCl), Sodium Phosphate monobasic (NaH2PO4-H2O), D-Glucose (Dextrose), Lipoic Acid and Sodium Pyruvate.

The skilled person may choose from the commercially available minimum essential culture medium the one which is the more appropriated to carry out the cell culture according to the invention.

Advantageously, the invention relates to the cell culture as defined above, wherein said endocrine cell is a placental cell line.

According to the invention a cell line is a permanently established cell culture that will proliferate indefinitely given appropriate fresh medium and space. Cell culture and cell lines have assumed an important role in studying physiological, pathophysiological, and differentiation processes of specific cells. It allows the examination of stepwise alterations in the structure, biology, and genetic makeup of the cell under controlled environments.

Advantageously, the invention relates to the cell culture as defined above, cytotrophoblastic placental cell, that produces hormones.

The cytotrophoblast (or layer of Langhans) is the inner layer of the trophoblast. It is interior to the syncytiotrophoblast and external to the wall of the blastocyst in a developing embryo.

Cytotrophoblast is a part of placenta. It is considered to be the trophoblastic stem cell because the layer surrounding the blastocyst remains while daughter cells differentiate and proliferate to function in multiple roles. There are two lineages that cytotrophoblastic cells may differentiate through: fusion and invasive. The fusion lineage yields syncytiotrophoblast and the invasive lineage yields interstitial cytotrophoblast cells. The ability of cytotrophoblast cells to produce hCG, progesterone, estrogen, cGnRH and beta-endorphin in vitro has been demonstrated earlier. Thus, cytotrophoblastic cells are good candidate for evaluating the effect of endocrine disruptor compounds on hormone production, and to determine if an unknown compound can be classified as an endocrine disruptor.

Advantageously, the invention relates to the cell culture as defined above, wherein the endocrine cell, especially the placental cell, is strictly adherent to a support onto which the endocrine cells, especially the placental cells, are cultured.

In order to efficiently use the cell culture according to the invention, it is advantageous that the endocrine cell contained in the cell culture mentioned above harbors adherent properties, i.e. to be able to interact with the support where the culture is carried out. This allows in particular to reuse the cell culture after exposure to a compound, by simple washes. in order to remove the presence of a compound that was used in order for instance to evaluate if it can be classified as an endocrine disruptor, and to use the same culture (and the same cells) to evaluate another compound.

Advantageously, the invention relates to the cell culture as defined above, wherein said endocrine cell is the placental cell line JEG-3, in particular the placental cell line deposited at ATCC under the number ATCC® HTB-36™ JEG3 cell line was initially derives from a human choriocarcinoma, and was characterized to secrete Human chorionic gonadotrophin (hCG), somatomammotrophin, progesterone and other hormones such as estradiol and polypepditic hormone such as human hPL.

Clone HTB-36 was deposited at the American collection of Cell Culture ATCC, and is available to the public. this clone is a hypertriploid human cell line. The modal chromosome number is 71, occurring at 34%, and polyploidy at 2.6%. The t(4;11)(p15;q13), i(13q), t(10p15q), del(18)(q21), and 6 other markers are common to most cells, and two other markers are found in some. Giant satellites are seen in one N14, and two N22. N2, N5, and N9 have 4 copies, and N7, N13, N18, N21 and X a single copy. A single Y chromosome is detected by Q-band examination.

The recommended culture conditions are: Eagle's Minimum Essential Medium, supplemented with fetal bovine serum to a final concentration of 10%. Antibiotics and glutamine may also be added for cell expansion.

When preparing the cell culture according to the invention, JEG-3 cells, and in particular clone HTB-36, are expended in the above mentioned medium, then washed many times with a free-serum medium, and then placed in a minimal essential medium supplemented with 1.5 to 3.5% weight of serum compared to the total weight of the culture medium.

The invention relates to a cell culture comprising:
a human endocrine placental cell; and
a culture medium consisting of minimal essential nutriments and serum, wherein said serum represents from 1.5 to 3.5% weight, preferably about 2.5% weight compared to the total weight of the culture medium.

The cell culture defined above does not contains antibiotics, and is not a cell culture containing rich culture medium such as OPTIMUM medium.

The purpose of this specific culture medium is to limit interference of the proteins and antibiotics such that the cell is directly in contact with the compound liable to be an endocrine disruptor.

Advantageously the invention relates to the cell culture defined above, wherein said endocrine placental cell is a placental cell line.

Advantageously the invention relates to the cell culture defined above, wherein the endocrine cell is a cytotrophoblastic placental cell.

Advantageously the invention relates to the cell culture defined above, wherein the endocrine cell is strictly adherent to a support onto which the endocrine cells are cultured.

Advantageously the invention relates to the cell culture defined above, wherein said endocrine cell is the placental cell line JEG-3, in particular the placental cell line deposited at ATCC under the number ATCC HTB-36.

The invention also relates to the use of the cell culture according to the above definition, for determining, in vitro, if a compound is an endocrine disruptor,
said compound being an endocrine disruptor when
it modulates the expression level of at least one hormone of a set of four hormones, wherein the set comprises a progesterone hormone and a polypeptidic hormone or its derivatives,
and
it modulates the expression and/or the activation of a P2X7 membrane receptor protein

The invention also relates to the use of the cell culture as defined above, for identifying, in vitro, if a compound is an endocrine disruptor

The invention also relates to the use of a cell culture as defined above, comprising:
a endocrine cell, preferably a placental cell; and
a culture medium consisting of minimal essential nutriments and serum,
wherein said serum represents from 1.5 to 3.5% weight compared to the total weight of the culture medium,
for identifying, in particular in vitro, if a compound is an endocrine disruptor compound, or disruptor compound.

The inventors have identified that the above mentioned cell culture is very useful to determine if a compound is classified as an endocrine disruptor, and thus should be withdrawn from commercialization for instance for incorporation in food stuff.

Moreover, the inventors identified that endocrine disruptors induce in the above mentioned cell culture, i.e. in endocrine cell cultured in a specific low serum containing culture media, inflammatory cell death via the pyroptosis pathway, this pathway being activated by P2X7 receptor.

Pyroptosis is known to be a form of inflammatory programmed cell death, triggered by various pathological stimuli such as stroke, heart attack, cancer, and microbial infections. Pyroptosis is fundamentally distinct from other cell death pathways by its dependency on caspase-1.

Pyroptosis is characterized by many features such as
cell lysis through cell swelling,
DNA cleavage, or DNA damages, but the nuclear integrity and oligonucleosomal DNA fragmentation are not observed,
activation of the inflammasome pathway—The inflammasome is a cytosolic multimeric signalling complex that coordinates the activation of an immune response against invading pathogens. Activation of the inflammasome subsequently leads to processing and activation of caspase-1—and
release of activated IL-1β and IL-18 cytokines.

It is more advantageous that the invention relates to the use of a cell culture as defined above, comprising:
a cytotrophobastic placental cell, preferably a JEG-3 cell line; and
a culture medium consisting of minimal essential nutriments and serum,
wherein said serum represents from 1.5 to 3.5% weight compared to the total weight of the culture medium,
for identifying, in particular in vitro, if a compound is an endocrine disruptor compound, or disruptor compound.

More advantageously, the invention relates to the use of a cell culture comprising:
the JEG-3 ATCC® HTB-36™ cell line; and
a culture medium consisting of minimal essential nutriments and serum,
wherein said serum represents from 1.5 to 3.5% weight compared to the total weight of the culture medium,
for identifying, in particular in vitro, if a compound is an endocrine disruptor compound, or disruptor compound.

The invention also relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring
   i—either the expression level and/or the activity of a P2X7 membrane receptor protein, to obtain respectively a measured P2X7 receptor protein expression level and/or a measured P2X7 receptor protein activity;
   ii—or the activation of inflammasome pathway, to obtain a measured inflammasome activity;
   iii—or both;
d—comparing
   the measured P2X7 receptor protein expression level to a control expression level of the P2X7 receptor protein determined by measuring the expression level of a P2X7 receptor protein of said endocrine cell contacted with a compound known to not be an endocrine disruptor, and/or
   the measured P2X7 receptor protein activity to a control activity of the P2X7 receptor protein determined by measuring the activity of the P2X7 receptor protein of said endocrine cell contacted with a compound known to not be an endocrine disruptor; and/or
   the measured inflammasome activity to a control activity of the inflammasome pathway determined in said endocrine cells contacted with a compound known to not be an endocrine disruptor;
      and
e—concluding
   if at least one of the measured P2X7 receptor protein, the measured P2X7 receptor protein activity, and the measured inflammasome activity is higher than the respective control P2X7 receptor protein expression, the control P2X7 receptor protein activity and control inflammasome activity, then the compound is an endocrine disruptor,
   otherwise, the compound is not an endocrine disruptor.

The inventors have discovered that measuring the expression level activity or the activation of inflammasome pathway in the cell of the cell culture defined above, allows to assess if a compound that modulate hormone expression is an accurate endocrine disruptor or not.

The invention also relates to a method for in vitro determining if a compound is an endocrine disruptor, said method comprising
a—contacting a compound liable to be an endocrine disruptor with a cell culture the cell culture comprising a human endocrine placental cell cultured in a culture medium, the culture medium comprising minimal essential nutriments and a serum, wherein said serum represents from 1.5 to 3.5% weight compared to the total weight of the culture medium of the cell culture, then
b—measuring, in said culture medium contacted with a compound liable to be an endocrine disruptor, an expression level of a set of four hormones, the set comprising a first, a second, a third and a fourth hormone, each of the first, second, third and fourth hormone being secreted by the human endocrine placental cell, to obtain a measured expression level of the first, second, third and fourth hormones,
   wherein the set comprises a progesterone hormone and a polypeptidic hormone or its derivatives, and
   comparing the measured expression level of each of the first, second, third and fourth hormone with a respective control expression level of each of the first, second, third and fourth hormone, the control expression level of each of the first, second, third and fourth hormone being measured in a culture medium from a cell culture containing a human endocrine placental cell which is not contacted with the compound liable to be an endocrine disruptor or which is contacted with a compound known not to be an endocrine disruptor,
c—measuring, in the human endocrine placental cell from the cell culture contacted with the compound liable to be an endocrine disruptor, an expression level and/or an activity, of a P2X7 membrane receptor protein, to obtain a measured expression level and/or activity of the P2X7 membrane receptor protein, and
   1. comparing the measured expression level and/or activity of the P2X7 membrane receptor protein with a control expression level and/or activity of the P2X7, said control expression level and/or activity of the P2X7 being measured in a human endocrine placental cell of a cell culture which is not contacted with the compound liable to be an endocrine disruptor or which is contacted with a compound known not to be an endocrine disruptor,
d—concluding that:
   i if the measured expression level of at least one of the first, second, third and fourth hormone is significatively different from the respective control of each of the first, second, third and fourth hormone, and the measured expression level and/or activity of the P2X7 membrane receptor protein is significatively different from its respective control, then the compound is an endocrine disruptor;
   ii if the measured expression level of at least one of the first, second, third and fourth hormone is significatively different from the respective control of each of the first, second, third and fourth hormone, but the measured expression level and/or activity of the P2X7 membrane receptor protein is not significatively different from its respective control, then it is not excluded that the compound is endocrine disruptor,
   iii if only the measured expression level of the measured expression level and/or activity of the P2X7 membrane receptor protein is significatively different from the respective control, then the compound is not an endocrine disruptor.

In other words, the invention provide a very efficient method allowing to determine if a compound is an endocrine disruptor by combining
measuring the variation of secretion of specifics hormones,
measuring the expression and/or activity of the receptor P2X7, and
by using a human placental cell that secrete specific hormones, which is very sensitive to the endocrine disruptor, because it is cultures in conditions wherein the serum is in an amount sufficient to maintain the cell alive, but that do not interfere with the compounds (sequestration of the compound by the large amount of proteins contained in the serum).

When both the hormones and the P2X7 receptors are simultaneously significantly modified compared to a reference or a control, then it is established that the compound is an endocrine disruptor and has to be withdrawn from the market.

When only the hormones are modified, which correspond to the currently available test, it is not possible to definitely state that the compound is an endocrine disruptor, because to meet this definition, a damage has to be observed in cells.

When only P2X7 is modified compared to a control, then the compound is not an endocrine disruptor, since these kinds of compounds have to modify hormone expression.

Advantageously, the invention relates to the method as defined above, the method further comprises:
measuring
   the activation of inflammasome pathway, in the human endocrine placental cell from the cell culture contacted with the compound liable to be an endocrine disruptor to obtain a measured inflammasome activity; or
   mitochondrial activity in the human endocrine placental cell from the cell culture contacted with the compound liable to be an endocrine disruptor, to obtain a measured mitochondria activity,
   or both;
comparing
   the measured inflammasome activity to a control activity of the inflammasome pathway, said control activity of the inflammasome pathway being measured in a human endocrine placental cell of a cell culture which is not contacted with the compound liable to be an endocrine disruptor or which is contacted with a compound known not to be an endocrine disruptor;
and/or
   the measured mitochondria activity to a control activity of the mitochondria, said control activity of control activity of the mitochondria being measured in a human endocrine placental cell of a cell culture which is not contacted with the compound liable to be an endocrine disruptor or which is contacted with a compound known not to be an endocrine disruptor; and
d—concluding that,
   if the measured inflammasome activity or the mitochondria activity is significantly different from the respective control inflammasome activity and the control mitochondria activity, then the compound is an endocrine disruptor, and
   otherwise, it is not excluded that the compound is endocrine disruptor.

If P2X7 is not modified, or not significantly modified, it can be stated that the compound tested is an endocrine disruptor when at least one of the mitochondria activity or the inflammasome activity is modified compared to a control.

Mitochondria activity can be measured by many technics well known in the art, for instance by measuring the transmembrane potential and its variation, or by measuring activity of some mitochondrial enzymes.

More advantageously, the invention relates to the method as defined above, wherein the method further comprises:
measuring the presence of DNA damages in said endocrine cells, to obtain a measured DNA fragmentation,
comparing the measured DNA damages to a control DNA damage, said control DNA damage being measured in a human endocrine placental cell of a cell culture which is not contacted with the compound liable to be an endocrine disruptor or which is contacted with a compound known not to be an endocrine disruptor, and
concluding that:
   when it is not excluded that the compound is endocrine disruptor
      if the measured DNA damages is significantly different from the control DNA fragmentation, then the compound is an endocrine disruptor having genotoxic effects, and
      if the measured DNA damages is not significantly different from the control DNA damage, then it is not excluded that the compound is endocrine disruptor, and
   when the compound is an endocrine disruptor,
      if the measured DNA damages is significantly different from the control DNA fragmentation, then the compound is an endocrine disruptor having genotoxic effects, then the compound is an endocrine disruptor, and
      if the measured DNA damages is not significantly different from the control DNA fragmentation, then the compound is an endocrine disruptor having no genotoxic effects.

By measuring DNA damages, it can be determined if a compound is an endocrine disruptor, and to establish that this compound is a genotoxic agent.

More advantageously, the invention relates to the method as defined above, wherein the method further comprises:
measuring the expression in the culture medium of said endocrine cells of hormones induced upon carcinogenic stimulation, to obtain a measured carcinogenic stimulation,
comparing the measured carcinogenic stimulation to a control carcinogenic stimulation, said control carcinogenic stimulation being measured in a human endocrine placental cell of a cell culture which is not contacted with the compound liable to be an endocrine disruptor or which is contacted with a compound known not to be an endocrine disruptor, and
concluding that
   when it is not excluded that the compound is endocrine disruptor
      if the measured carcinogenic stimulation is significantly different from the control carcinogenic stimulation, then the compound is an endocrine disruptor having carcinogenic effects, and
if the measured carcinogenic stimulation is not significantly different from the control carcinogenic stimulation, then it is not excluded that the compound is endocrine disruptor, and
   when the compound is an endocrine disruptor,
      if the measured carcinogenic stimulation is significantly different from the control carcinogenic stimulation, then the compound is an endocrine disruptor having carcinogenic effects, and
      if the measured carcinogenic stimulation is not significantly different from the control carcinogenic stimulation, then the compound is an endocrine disruptor having no carcinogenic effects.

By measuring carcinogenic stimulations, it can be determined if a compound is an endocrine disruptor, and to establish that this compound is a carcinogenic agent, i.e. an agent inducing cancer.

More advantageously, the invention relates to the method as defined above, the method further comprises:
measuring the activity of the aromatase enzyme of said endocrine cells, to obtain a measured aromatase activity,
comparing the measured aromatase activity to a control aromatase activity, said control aromatase activity being measured in a human endocrine placental cell of a cell culture which is not contacted with the compound liable to be an endocrine disruptor or which is contacted with a compound known not to be an endocrine disruptor, and
d—concluding that
   when it is not excluded that the compound is endocrine disruptor
      if the measured aromatase activity is significantly different from the control aromatase activity, then the compound is an endocrine disruptor having effects on fertility, and
      if the measured aromatase activity is not significantly different from the control aromatase activity, it is not excluded that the compound is endocrine disruptor, and
   when the compound is an endocrine disruptor,
      if the measured aromatase activity is significantly different from the control aromatase activity, then the compound is an endocrine disruptor having effects on fertility, and
      if the measured aromatase activity is not significantly different from the control aromatase activity, then the compound is an endocrine disruptor having no effects on fertility.

By measuring aromatase activation, it can be determined if a compound is an endocrine disruptor, and to establish that this compound will have some effects on fertility of individuals submitted to it.

More advantageously, the invention relates to the method as defined above, wherein there is a significant difference when the measured and the control values differ of +/−15%.

For measuring the difference between the measured and the control data, the skilled person will carry out statistical analysis, according to his common knowledge. When the difference is higher than 15% the difference is considered to be significant.

For instance, if an hormone is expressed at a level 1 in the control sample, and at a level of 1.16, or 0.84 in a culture medium suspected to be an endocrine disruptor, the difference is higher than 15% (decrease of 15% or increase of 15%) this difference is considered to be significant. This example is not limitative and is only give to clarify the purpose of the invention.

More advantageously, the invention relates to the method as defined above, wherein the peptidic hormones are beta Chorionic gonadotropin hormone or RhCG, or one of its derivative, such that a glycosyltated RhCG, and Human Placental Lactogen or hPL.

In one advantageous embodiment, the invention relates to the above defined method, wherein the activation of inflammasome pathway is measured by evaluating caspase-1 protein activity, and/or IL1β expression and/or secretion.

This method is based on the use of the cell culture medium defined above.

In a first step of the method, the cells, which are preferably JEG-3 cells are cultures in a medium supplemented with 1.5 to 3.5% of serum by weight compared to the total weight of the culture medium.

Then a compound that it is suspected to be an endocrine disruptor is contacted with the cell culture such that the compound can possibly affect the secretion of hormones by the cells of the cell culture.

If the compound does not affect the secretion of hormones, it is not proceeded further with this method because the compound is not an endocrine disruptor.

If the compound affect secretion of hormones, that can be evaluated by well-known method disclosed in the art, the next step consists to evaluate, either the expression or the activity of the P2X7 receptor at the cell membrane of the calls of the cell culture, and/or evaluate the activation of inflammasome pathway.

The P2X7 receptor is a trimeric ion channel gated by extracellular adenosine 5′-triphosphate supporting Na+ and Ca2+ influx into and K+ efflux out of the cell cytoplasm. This receptor mediates the formation of membrane pores upon activation by extracellular adenosine triphosphate, leading to activation of some intracellular signaling pathways associated with numerous physiological and pathophysiological processes from migration to cell death, through induction of the inflammatory cascade.

In order to evaluate the expression of the receptor P2X7, it is possible to carry out an immunodetection using fluorescent labelled antibodies in order detect the presence and/or the amount of receptor at the cell membrane. It is also possible to evaluate the expression by quantifying the mRNA amount coding for the receptor, for instance by using quantitative techniques such as RT-qPCR.

The activity of P2X7 can be measured by evaluating the opening of the pore constituted by the receptor. For instance, it is possible to use fluorescent proteins that cannot enter into cells when the receptor is not activated, but can enter when the receptor is activated. An example of such compounds is disclosed in the following Example 2, that discloses the use of YO-PRO-1 as disclosed in detail in Rat et al. J Biol Methods. 2017 Jan. 20; 4(1):e64, incorporated herein.

It is also possible to evaluate the activity of the inflammasome pathway. Inflammasomes, or inflammasome pathway, are multiprotein signalling platforms that control the inflammatory response and coordinate antimicrobial host defences. They are assembled by pattern-recognition receptors following the detection of pathogenic microorganisms and danger signals in the cytosol of host cells, and they activate inflammatory caspases to produce cytokines and to induce pyroptotic cell death.

Inflammasomes activate inflammatory caspases, cysteine-dependent aspartate-directed proteases, which promote the maturation of the cytokines interleukin-1B3 (IL-1B) and IL-18. Analogous to the apoptosome, which activates apoptotic cascades, the inflammasome pathway activates a pyroptotic inflammatory cascade. Once active, the inflammasome binds to pro-caspase-1 (the precursor molecule of caspase-1), either homotypically via its own caspase activation and recruitment domain (CARD) or via the CARD of the adaptor protein ASC which it binds to during inflammasome formation. In its full form, the inflammasome appositions together many p45 pro-caspase-1 molecules, inducing their autocatalytic cleavage into p20 and p10 subunits. Caspase-1 then assembles into its active form consisting of two heterodimers with a p20 and p10 subunit each. Once active, it can then carry out a variety of processes in response to the initial inflammatory signal.

In order to measure the activity of inflammasome, it is possible to measure the activity of caspase-1 protein, and/or secretion of IL-1β.

All the data collected are then compared to a reference, i.e a reference expression of P2X7, a reference activation of P2X7, and a reference activation of inflammasome. These references data are obtained by contacting the cell culture with a compound that is known to not be an endocrine disruptor. For instance, water, solvent that are used to solubilize the compounds liable to be endocrine disruptors, can be used.

In the last step of the process according to the invention, it is evaluated if the measured activation/expression of P2X7 or activation of inflammasome is significantly different to the reference. If all the measures are close to the control, then the compound can be considered not to be an endocrine disruptor. Otherwise, if one of the measures is significantly different from the control, then the compound can be classified as an endocrine disruptor.

Advantageously, the invention relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring the expression level of a P2X7 membrane receptor protein, to obtain a measured P2X7 receptor protein expression level;
d—comparing the measured P2X7 receptor protein expression level to a control expression level of the P2X7 receptor protein determined by measuring the expression level of a P2X7 receptor protein of said endocrine cell contacted with a compound known to not be an endocrine disruptor,
e—concluding
   if the measured P2X7 receptor protein, is higher than the control P2X7 receptor protein expression, then the compound is an endocrine disruptor,
   otherwise, the compound is not an endocrine disruptor.

Advantageously, the invention relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring the activity of a P2X7 membrane receptor protein, to obtain a measured P2X7 receptor protein activity;
d—comparing the measured P2X7 receptor protein activity to a control activity of the P2X7 receptor protein determined by measuring the activity of the P2X7 receptor protein of said endocrine cell contacted with a compound known to not be an endocrine disruptor;
e—concluding
if the measured P2X7 receptor protein activity, is higher than the control P2X7 receptor protein activity, then the compound is an endocrine disruptor,
otherwise, the compound is not an endocrine disruptor.

Advantageously, the invention also relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring the activation of inflammasome pathway, to obtain a measured inflammasome activity;
d—comparing the measured inflammasome activity to a control activity of the inflammasome pathway determined in said endocrine cells contacted with a compound known to not be an endocrine disruptor;
and
e—concluding
   if the measured inflammasome activity is higher than the control inflammasome activity, then the compound is an endocrine disruptor,
   otherwise, the compound is not an endocrine disruptor.

More advantageously, the invention relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring
   i—the expression level and the activity of a P2X7 membrane receptor protein, to obtain respectively a measured P2X7 receptor protein expression level and/or a measured P2X7 receptor protein activity;
   ii—and the activation of inflammasome pathway, to obtain a measured inflammasome activity;
d—comparing
   the measured P2X7 receptor protein expression level to a control expression level of the P2X7 receptor protein determined by measuring the expression level of a P2X7 receptor protein of said endocrine cell contacted with a compound known to not be an endocrine disruptor,
and
   the measured P2X7 receptor protein activity to a control activity of the P2X7 receptor protein determined by measuring the activity of the P2X7 receptor protein of said endocrine cell contacted with a compound known to not be an endocrine disruptor; and
   the measured inflammasome activity to a control activity of the inflammasome pathway determined in said endocrine cells contacted with a compound known to not be an endocrine disruptor;
and
e—concluding
   if at least one of the measured P2X7 receptor protein, the measured P2X7 receptor protein activity, and the measured inflammasome activity is higher than the respective control P2X7 receptor protein expression, the control P2X7 receptor protein activity and control inflammasome activity, then the compound is an endocrine disruptor,
   otherwise, the compound is not an endocrine disruptor.

More advantageously, the invention relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring
   i—the expression level or the activity of a P2X7 membrane receptor protein, to obtain respectively a measured P2X7 receptor protein expression level and/or a measured P2X7 receptor protein activity;
   and the activation of inflammasome pathway, to obtain a measured inflammasome activity;
d—comparing
   the measured P2X7 receptor protein expression level to a control expression level of the P2X7 receptor protein determined by measuring the expression level of a P2X7 receptor protein of said endocrine cell contacted with a compound known to not be an endocrine disruptor, or
   the measured P2X7 receptor protein activity to a control activity of the P2X7 receptor protein determined by measuring the activity of the P2X7 receptor protein of said endocrine cell contacted with a compound known to not be an endocrine disruptor; and
   the measured inflammasome activity to a control activity of the inflammasome pathway determined in said endocrine cells contacted with a compound known to not be an endocrine disruptor;
and
e—concluding
   if at least one of the measured P2X7 receptor protein, the measured P2X7 receptor protein activity, and the measured inflammasome activity is higher than the respective control P2X7 receptor protein expression, the control P2X7 receptor protein activity and control inflammasome activity, then the compound is an endocrine disruptor,
   otherwise, the compound is not an endocrine disruptor.

Advantageously, the invention relates to the method defined above, further comprising before step e, steps of:
c1—measuring the presence of DNA damages in said endocrine cells, to obtain a measured DNA fragmentation
d1—comparing the measured DNA damages to a control DNA damages obtained in said endocrine cell contacted with a compound know to be an endocrine disruptor, and
e—concluding that
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured DNA damages is higher than the control DNA fragmentation, then the compound is an endocrine disruptor having genotoxic effects, and
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured DNA damages is lower than or equal to the control DNA fragmentation, then the compound is an endocrine disruptor having no genotoxic effects,
otherwise, the compound is not an endocrine disruptor.

In other words, advantageously, the invention relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring
   i—either the expression level and/or the activity of a P2X7 membrane receptor protein, to obtain respectively a measured P2X7 receptor protein expression level and/or a measured P2X7 receptor protein activity;
   ii—or the activation of inflammasome pathway, to obtain a measured inflammasome activity;
   iii—or both;
   iv—and measuring the presence of DNA damages in said endocrine cells, to obtain a measured DNA damages;
d—comparing
   the measured P2X7 receptor protein expression level to a control expression level of the P2X7 receptor protein determined by measuring the expression level of a P2X7 receptor protein of said endocrine cell contacted with a compound known to not be an endocrine disruptor, and/or
   the measured P2X7 receptor protein activity to a control activity of the P2X7 receptor protein determined by measuring the activity of the P2X7 receptor protein of said endocrine cell contacted with a compound known to not be an endocrine disruptor; and/or
   the measured inflammasome activity to a control activity of the inflammasome pathway determined in said endocrine cells contacted with a compound known to not be an endocrine disruptor;
and
   comparing the measured DNA damages to a control DNA damages obtained in said endocrine cell contacted with a compound know to be an endocrine disruptor;
and
e—concluding that
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured DNA damages is higher than the control DNA fragmentation, then the compound is an endocrine disruptor having genotoxic effects, and
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured DNA damages is lower than or equal to the control DNA fragmentation, then the compound is an endocrine disruptor having no genotoxic effects,
   otherwise, the compound is not an endocrine disruptor.

The inventors have shown that it is also possible to determine if an endocrine disruptor identified by the method as defined above could have genotoxic effects.

For this purpose it is possible to detect, when assessing the status of the compound, possible DNA damages in the cells of the cell culture.

Evaluation of DNA damages is well known in the art, and can be easily carried out by the skilled person.

In the current invention DNA damages corresponds to double strand brake of the DNA molecules contained in a cell, or a genotoxic alteration of DNA.

Advantageously, the invention relates to the method defined above, further comprising before step e, steps of
c2—measuring the expression in the culture medium of said endocrine cells of hormones induced upon carcinogenic stimulation, to obtain a measured carcinogenic stimulation,
d2—comparing the measured carcinogenic stimulation to a control carcinogenic stimulation obtained in said endocrine cells contacted with a compound know to be an endocrine disruptor, and
e—concluding that
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured carcinogenic stimulation is higher than the control carcinogenic stimulation, then the compound is an endocrine disruptor having carcinogenic effects, and
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured carcinogenic stimulation is lower than or equal to the control carcinogenic stimulation, then the compound is an endocrine disruptor having no carcinogenic effects,
   otherwise, the compound is not an endocrine disruptor.

In other words, advantageously, the invention relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring
   i—either the expression level and/or the activity of a P2X7 membrane receptor protein, to obtain respectively a measured P2X7 receptor protein expression level and/or a measured P2X7 receptor protein activity;
   ii—or the activation of inflammasome pathway, to obtain a measured inflammasome activity;
   iii—or both;
   iv—and measuring the expression in the culture medium of said endocrine cells of hormones induced upon carcinogenic stimulation, to obtain a measured carcinogenic stimulation;
d—comparing
   the measured P2X7 receptor protein expression level to a control expression level of the P2X7 receptor protein determined by measuring the expression level of a P2X7 receptor protein of said endocrine cell contacted with a compound known to not be an endocrine disruptor, and/or
   the measured P2X7 receptor protein activity to a control activity of the P2X7 receptor protein determined by measuring the activity of the P2X7 receptor protein of said endocrine cell contacted with a compound known to not be an endocrine disruptor; and/or
   the measured inflammasome activity to a control activity of the inflammasome pathway determined in said endocrine cells contacted with a compound known to not be an endocrine disruptor;
and
   comparing the measured carcinogenic stimulation to a control carcinogenic stimulation obtained in said endocrine cells contacted with a compound know to be an endocrine disruptor;
and
e—concluding that
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured carcinogenic stimulation is higher than the control carcinogenic stimulation, then the compound is an endocrine disruptor having carcinogenic effects, and
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured carcinogenic stimulation is lower than or equal to the control carcinogenic stimulation, then the compound is an endocrine disruptor having no carcinogenic effects,
   otherwise, the compound is not an endocrine disruptor.

The inventors have shown that it is also possible to determine if an endocrine disruptor identified by the method as defined above could have carcinogenic effects.

For this purpose it is possible to detect, when assessing the status of the compound, possible secretion of specific hormones that are stimulated and secreted upon stimulation by a carcinogenic compound.

It is for instance possible to evaluate the glycosylation profile of some hormones such as hCG. Indeed, specific glycosylations have been identified in human when submitted to carcinogens.

Advantageously, the invention relates to the method defined above, further comprising before step e, steps of
c3—measuring the activity of the aromatase enzyme of said endocrine cells, to obtain a measured aromatase activity,
d3—comparing the measured aromatase activity to a control aromatase activity obtained in said endocrine cells contacted with a compound know to be an endocrine disruptor, and
e—concluding that
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured activity is higher than the control aromatase activity, then the compound is an endocrine disruptor having effects on fertility, and
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured activity is lower than or equal to the control aromatase activity, then the compound is an endocrine disruptor having no effects on fertility,
   otherwise, the compound is not an endocrine disruptor.

In other words, advantageously, the invention relates to a method for determining, preferably in vitro, if a compound is an endocrine disruptor, said method comprising
a—providing the cell culture as defined above,
b—contacting the endocrine cell of the cell culture with a compound liable to be an endocrine disruptor, said compound modulating hormone production by endocrine cells,
c—measuring
   i—either the expression level and/or the activity of a P2X7 membrane receptor protein, to obtain respectively a measured P2X7 receptor protein expression level and/or a measured P2X7 receptor protein activity;
   ii—or the activation of inflammasome pathway, to obtain a measured inflammasome activity;
   iii—or both;
   iv—and measuring the activity of the aromatase enzyme of said endocrine cells, to obtain a measured aromatase activity;
d—comparing
   the measured P2X7 receptor protein expression level to a control expression level of the P2X7 receptor protein determined by measuring the expression level of a P2X7 receptor protein of said endocrine cell contacted with a compound known to not be an endocrine disruptor, and/or
   the measured P2X7 receptor protein activity to a control activity of the P2X7 receptor protein determined by measuring the activity of the P2X7 receptor protein of said endocrine cell contacted with a compound known to not be an endocrine disruptor; and/or
   the measured inflammasome activity to a control activity of the inflammasome pathway determined in said endocrine cells contacted with a compound known to not be an endocrine disruptor;
and
   comparing the measured aromatase activity to a control aromatase activity obtained in said endocrine cells contacted with a compound know to be an endocrine disruptor;
and
e—concluding that
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured activity is higher than the control aromatase activity, then the compound is an endocrine disruptor having effects on fertility, and
   if at least one of the measured P2X7 receptor protein, the measured P2X7 receptor protein activity, and the measured inflammasome activity is higher than the respective control P2X7 receptor protein, the control P2X7 receptor protein activity and control inflammasome activity, and the measured activity is lower than or equal to the control aromatase activity, then the compound is an endocrine disruptor having no effects on fertility,
   otherwise, the compound is not an endocrine disruptor.

The inventors have shown that it is also possible to determine if an endocrine disruptor identified by the method as defined above could have some effects on fertility.

For this purpose it is possible to detect, when assessing the status of the compound, activity of aromatase.

Aromatase (CYP19A, EC 1.14.14.14) is a member of the cytochrome P450 monooxidase (CYP) family of microsomal xenobiotic metabolism enzymes. Aromatase plays a critical role in steroidogenesis, catalyzing the conversion of androgenic hormones into estrogens. The enzyme is expressed in high levels in reproductive tissues, placenta, brain and adipose tissue and is responsible for mammalian sexual dimorphism and development of secondary sexual characteristics.

Many kits for detecting activity of aromatase are available, and the skilled person can easily choose the more appropriated.

More advantageously, the invention relates to the method defined above, wherein the compound liable to be an endocrine disruptor induces a variation of hormones expression of +/−20% compared to the hormone expression in absence of an endocrine disruptor.

More advantageously, the invention relates to the method defined above, wherein the set of 4 hormones comprises: estrogen, progesterone or polypeptidic hormones, such as human placental lactogen (hPL) and human chorionic gonadotropin, beta polypeptide (RhCG).

Human placental lactogen (hPL), also called human chorionic somatomammotropin (HCS), is a polypeptide placental hormone. Its structure and function are similar to those of human growth hormone. hPL modifies the metabolic state of the mother during pregnancy to facilitate the energy supply of the fetus. hPL is secreted by the syncytiotrophoblast during pregnancy.

β hCG is also a polypeptide placental hormone, which is initially secreted by the syncytiotrophoblast. It is also advantageous to measure the expression of a glycosylated β hCG hormone, since this hormone is naturally expressed only in the first days of human placenta, but is deregulated when the endocrine cell is exposed to an carcinogen endocrine disruptor.

More advantageously, the invention relates to the method defined above, wherein the activation of inflammasome pathway is measured by evaluating caspase-1 protein activity, and/or IL1β expression and/or secretion.

Caspase-1 protein activity is easy to evaluate by using different kits known in the art. For instance, caspase-1 activity can be measured by detecting the cleavage of a substrate YVAD-AFC (AFC: 7-amino-4-trifluoromethyl coumarin). YVAD-AFC emits blue light (Em=400 nm); upon cleavage of the substrate by caspase-1 or related caspases, free AFC emits a yellow-green fluorescence (Ex/Em=400/505 nm), which can be quantified using a fluorometer or a fluorescence microtiter plate reader. Comparison of the fluorescence from a treated sample with an untreated control allows determination of the fold increase in caspase-1 activity.

IL1β expression and/or secretion can be evaluated by using immunological means, either directly in the culture medium for a secreted IL R, or directly in cells.

Expression of IL1β can also be measured by evaluating the gene expression, for instance by using RT-PCR protocols using specifics oligonucleotides and probes.

The invention also relates to a kit comprising:
a cell culture as defined above,
at least a compound known to not be an endocrine disruptor, and
at least a means for measuring a P2X7 receptor protein expression and/or activation.

The invention also relates to a kit comprising:
a cell culture as defined above,
at least a compound known to be an endocrine disruptor, such as Bisphenol A, and
at least a means for measuring a P2X7 receptor protein expression and/or activation.

The invention also relates to a kit comprising:
a cell culture as defined above,
at least a compound known to be an endocrine disruptor, such as Bisphenol A, and at least a compound known not to be an endocrine disruptor, in particular culture medium,
at least a means for measuring a P2X7 receptor protein expression and/or activation.

Advantageously, the above-mentioned kit comprises:
a JEG-3 cell line, and a culture medium comprising minimum essential nutriments and serum in an amount as defined above,
at least a compound known to not be an endocrine disruptor, and
at least a means for measuring a P2X7 receptor protein expression and/or activation, such as YO-PRO-1 compound.

Advantageously, the above-mentioned kit comprises:
a JEG-3 cell line, and a culture medium comprising minimum essential nutriments and serum in an amount as defined above,
at least a compound known to be an endocrine disruptor, such as Bisphenol A, and
at least a means for measuring a P2X7 receptor protein expression and/or activation, such as YO-PRO-1 compound.

Advantageously, the above-mentioned kit comprises:
a JEG-3 cell line, and a culture medium comprising minimum essential nutriments and serum in an amount as defined above,
at least a compound known to be an endocrine disruptor, such as Bisphenol A, and at least a compound known not to be an endocrine disruptor, and
at least a means for measuring a P2X7 receptor protein expression and/or activation, such as YO-PRO-1 compound.

The kit may also comprise, means for detecting DNA damages and/or means for detecting expression of hormones that are secreted upon carcinogen stimulation, and/or means for evaluating aromatase activity.

The invention also relates to a kit, as defined above, comprising:
a cell culture comprising a human placental endocrine cell, and
means for measuring the expression of four hormones secreted by placental cells, the four hormones comprising a progesterone hormone and a peptidic hormone or its derivatives secreted by placental cells,
and possibly means for measuring the expression and/or activation of the P2X7 receptor.

The invention will be better understood in light of the following figures and examples.

### BRIEF DESCRIPTION OF THE DRAWINGS

FIG. 1 represents a graph showing the proliferation of JEG-3 cells in culture medium supplemented with different FBS concentrations. JEG-3 cells were incubated with three different concentrations of FBS for 24 or 72 hours, cell count was conducted to quantify the effect of FBS on JEG-3 cell proliferation. Black: 10% FBS, dark grey: 2.5% FBS, light grey: 0% FBS. Y-axis: number of living cells (cells/mL); X-axis time (hours)

FIG. 2 represents photography showing the expression of CK7 in JEG-3 cells in 2.5% and 10% FBS.
A—Cells cultured with 2.5% FBS were stained with anti-CK7 antibody and then stained with Alexa Fluor 488.
B—Cells cultured with 2.5% FBS were stained DAPI used to stain DNA (blue).
C—Cells cultured with 10% FBS were stained with anti-CK7 antibody and then stained with Alexa Fluor 488.
D—Cells cultured with 10% FBS were stained DAPI used to stain DNA (blue). representative of at least 3 independent experiments.
E—Cells cultured with 10% FBS were stained with an isotype control and then stained with Alexa Fluor 488.
F—Cells cultured with 10% FBS were stained DAPI used to stain DNA (blue).

Data are representative of at least 3 independent experiments.

FIG. 3 is a graph showing the quantification of CK7 fluorescence using ImageJ software. Normalized CK7 fluorescence intensity was obtained by dividing green fluorescence intensity by blue fluorescence intensity to take into account the difference in cell numbers in the selected microscopic fields. Y-axis: Normalized CK7 intensity. A: control isotype; B: cells cultured with 2.5% FBS and C: cells cultured with 10% FBS.

FIG. 4 represents histograms showing the comparison of JEG-3 cell viability after incubation with SLS (A) or PFOA (B) in FBS 10% or FBS 2.5%. JEG-3 cells were incubated with SLS from 10 to 50 μg/mL or PFOA from 40 to 120 μM for 24 hours. Cell viability was determined using the neutral red assay. £££p<0.001 and ££££p<0.0001 compared to negative control in 10% FBS, ****p<0.0001 compared to negative control in 2.5% FBS (n=3). A: Y-axis: cell viability (%) and X-axis: SSL concentration (μg/mL); B: Y-axis: cell viability (%) and X-axis: PFOA concentration (μg/mL).

FIG. 5 represents graphs showing the evaluation of cell viability (in % −Y-axis) and chromatin condensation of JEG-Tox cells after incubation with apoptosis inducers for 24 hours (concentration in μg/mL for B, C, D, E, G and H or in % v/v for A and F). Cell viability and chromatin condensation were quantified using the Alamar blue and Hoechst 33342 assays, respectively. Dashed line: cell viability, solid line: chromatin condensation. *p<0.05, **p<0.01, ***p<0.001, and ****p<0.0001 compared to negative control (n=3). A: Ethanol; B: Quinalphos; C: Bisphenol F; D: 4-4′-DTT; E: BAC; F: Phenoxyethanol; G: Propylparaben and H: PFOA.

FIG. 6 represents Cell viability evaluated using the neutral red assay after bisphenol A (A), diethylstilbestrol (B), 4-tert-amylphenol (C), triclosan (E), propylparaben (F), butyl benzyl phthalate (G), dibutyl phthalate (H), DEHP (1) and 3-benzylidene camphor (J) incubation for 72 h on JEG-Tox cells. Black and white columns represent respectively solvent and control.

FIG. 7 represents P2X7 receptor activation evaluated after bisphenol A (A), diethylstilbestrol (B), 4-tert-amylphenol (C), triclosan (E), propylparaben (F), butyl benzyl phthalate (G), dibutyl phthalate (H), DEHP (1) and 3-benzylidene camphor (J) incubation for 72 h on JEG-Tox cells. ****p<0.0001, ***p<0.001, **p<0.01 and *p<0.05 compared to control. Black and white columns represent respectively solvent and control.

FIG. 8 represents Caspase-1 activity cells evaluated in JEG-Tox after incubation with bisphenol A (A), diethylstilbestrol (B), 4-tert-amylphenol (C), triclosan (E), propylparaben (F), butyl benzyl phthalate (G), dibutyl phthalate (H), DEHP (1) and 3-benzylidene camphor (J) for 72 h. ***p<0.001, **p<0.01 and *p<0.05 compared to control. Black and white columns represent respectively solvent and control.

FIG. 9 represents Caspase-9 activity cells evaluated in JEG-Tox after incubation triclosan (A), benzyl butyl phthalate (B), Dibutyl benzyl phthalate (C) DEHP (D) and 3-benzylidene camphor (E). ****p<0.0001 and ***p<0.001 compared to control. Black and white columns represent respectively solvent and control. Y-axis: Fold change in caspase-9 activity.

FIG. 10 represents a graph showing cell viability evaluated using the neutral red assay after Bisphenol A (BPA) incubation for 72 h on JEG-Tox cells.

FIG. 11 represents a graph showing cell viability evaluated using the neutral red assay after Bisphenol F (BPF) incubation for 72 h on JEG-Tox cells.

FIG. 12 represents a graph showing P2X7 receptor activation after BPA incubation for 72 h on JEG-Tox cells. ****p<0.0001, ***p<0.001 and **p<0.01 compared to control.

FIG. 13 represents a graph showing P2X7 receptor activation after BPF incubation for 72 h on JEG-Tox cells. ****p<0.0001, ***p<0.001 and **p<0.01 compared to control.

FIG. 14 represents a graph showing Caspase-1 activity evaluated in JEG-Tox cells after incubation with after BPA incubation for 72 h on JEG-Tox cells. ****p<0.0001, ***p<0.001 and *p<0.05 compared to control.

FIG. 15 represents a graph showing Caspase-1 activity evaluated in JEG-Tox cells after incubation with after BPF incubation for 72 h on JEG-Tox cells. ****p<0.0001, ***p<0.001 and *p<0.05 compared to control.

FIG. 16 represents a graph showing Caspase-9 activity evaluated in JEG-Tox cells after incubation with after BPA incubation for 72 h on JEG-Tox cells. ****p<0.0001, ***p<0.001 and *p<0.05 compared to control.

FIG. 17 represents a graph showing Caspase-9 activity evaluated in JEG-Tox cells after incubation with after BPF incubation for 72 h on JEG-Tox cells. ****p<0.0001, ***p<0.001 and *p<0.05 compared to control.

FIG. 18 represents a graph showing ROS production by using a H2DCF-DA assay after incubation during 48 h on JEG-Tox cells with after BPF incubation. ****p<0.0001 compared to control.

FIG. 19 represents a graph showing ROS production by using a H2DCF-DA assay after incubation during 48 h on JEG-Tox cells with after BPA incubation. ****p<0.0001 compared to control.

FIG. 20 is a schematic representation of P2X7 receptor activation upon stimulation of placental cells by endocrine disruptors, and the intracellular pathway consequently activated. The effects of endocrine disruptors are also shown in this schematic representation.

### EXAMPLES

### Example 1: JEG-Tox Model

The objective of the present example is to establish incubation conditions for JEG-3 placental cells to reveal pregnancy disorders induced by chemicals. To achieve this objective, the inventors first studied JEG-3 cells behavior in 2.5% serum compared to 10%, and second, they checked that JEG-3 cells are able to activation of P2X7 receptor and pyroptosis, as well as DNA damages and degenerative pathway in response to chemicals toxic for pregnant women.

Materials and Methods

1. Materials

All tested chemicals were purchased from Merck (Darmstadt, Germany) except ethanol (VWR Chemicals, Radnor, PA, USA) and perfluorooctanoic acid (ThermoFisher Scientific, Waltham, MA, USA).

All cell culture reagents were obtained from Gibco (Paisley, UK). 96-well microplates were purchased from Corning (Amsterdam, The Nederlands) and Nunc® Lab-Tek® II Chamber Slide™ system from Merck.

Antibodies were purchased from Merck (mouse anti-CK7 antibody) and ThermoFisher Scientific (Alexa Fluor 488 goat anti-mouse antibody and isotypic control). Fluorescent probes were obtained from ThermoFisher Scientific.

Fluorescence Resonance Energy Transfer (FRET) assays were performed with (HTRF® estradiol kit from Cisbio Biosassays, Codolet, France, and sandwich ELISA with MyBioSource, Vancouver, Canada human placental lactogen hormone kit and human hyperglycosylated Chorionic Gonadotropin hormone assay kit.

2. Cell Culture

The choriocarcinoma-derived JEG-3 cell-line (ATCC® HTB-36™, Manassas, VA, USA), was grown as recommended by ATCC: Minimum Essential Medium Eagle's medium supplemented with 10% fetal bovine serum, 2 mM of glutamine, 50 IU/mL of penicillin and 50 IU/mL of streptomycin. Cells were detached using trypsin, counted, and then seeded at 80,000 cells/mL in 96-well microplates (200 μL by well) and Nunc® Lab-Tek® II Chamber Slide™ system for immunostaining.

2.1. JEG-3 Cells Behavior in Culture Medium Supplemented with Different Concentrations of Serum

2.1.1. Impact of Fetal Bovine Serum (FBS) Concentration on Cell Proliferation

Cells were cultured in three different concentrations of FBS (using the same batch): 0%, 2.5% and 10%. At 24 and 72 hours, cells were detached using trypsin and then counted by the Countess™ II Automated Cell Counter (ThermoFisher Scientific, Waltham, MA, USA).

2.1.2. Cell Line Authentication by STR Analysis (Genetic Profile)

Cell line DNA was profiled by Short Tandem Repeat (STR). This technique also checks the lack of cellular cross-contamination (23). STR analysis was performed by the Human STR Profiling Cell Authentication Service of ATCC.

2.1.3. CK7 Immunostaining

The cytokeratin-7 (CK7) intermediate filament is an established marker of trophoblastic cells (14,24). 24 hours after seeding in culture medium supplemented with 2.5 or 10% FBS, JEG-3 cells were fixed in 4% paraformaldehyde for 20 min, permeabilized in 0.1% Triton X-100 for 10 min, saturated with a solution of 1% BSA and 0.1% Tween in PBS for 2 h, and then incubated overnight at 4° C. with mouse anti-CK7 antibody (196 μg/mL) diluted in PBS containing 1% BSA and 0.1% Tween 20. After washing, the cells were incubated with Alexa Fluor 488 goat anti-mouse antibody (4 μg/mL) diluted in PBS containing 1% BSA for 2 h at room temperature. Nuclei were stained with 300 nM DAPI for 5 min and Vectashield (Vector Laboratories, Burlingame, CA, USA) mounting medium was used for microscopy images (EVOS FL, ThermoFisher Scientific). Mouse IgG1 kappa clone P3.6.2.8.1 was used as an isotypic control to help differentiate non-specific background signal from specific antibody signal.

2.1.4. Hormone Release Quantitation

After 72 hours of incubation in cell culture medium supplemented with 2.5 or 10% FBS, microplates were centrifuged, and cell supernatants were collected. Estradiol was quantified in cell supernatants by Fluorescence Resonance Energy Transfer (FRET) technology (HTRF® Cisbio Biosassays) according to manufacturer's instructions. The detection limit of this assay is 20 μg/mL.

Human placental lactogen (hPI) hormone and human hyperglycosylated Chorionic Gonadotropin (hCG) hormone were measured by sandwich ELISA (MyBioSource) according to manufacturer's instructions. Sensitivities are <46.875 μg/mL and 39 μg/mL for hPI and hCG dosage, respectively.

2.1.5. Impact of Fetal Bovine Serum Concentration on Sodium Lauryl Sulfate (SLS) and Perfluorooctanoic Acid (PFOA) Cytotoxicity

Cells were incubated with sodium lauryl sulfate or perfluorooctanoic acid diluted in culture medium supplemented with either 2.5% or 10% FBS. After 24 hours, cell viability was evaluated using the neutral red assay. Neutral Red solution at 0.4% in water was diluted in culture medium with a ratio of 1:79 to give a final concentration of 50 μg/mL. Neutral Red was distributed in the plates for a 3-hour incubation time at 37° C. The cells were then rinsed with PBS to remove any remaining unincorporated dye. The dye was then released from the cells using a lysis solution (1% acetic acid, 50% ethanol and 49% H2O) and the fluorescence was measured (λex=540 nm, λem=600 nm) using Spark microplate fluorometer (Tecan, Mannedorf, Switzerland).

2.2. Apoptosis Evaluation after Incubation with Toxic Agents

2.2.1. Toxic Agents

Tested toxic agents for apoptosis evaluation were detailed in table 1. Solvents were evaluated alone to discriminate their potential effect (data not shown).

TABLE 1

| Family | Substance | formula |
|---|---|---|
| Bis-phenol | Bis-phenol A | 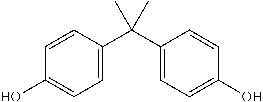 [SMILES: CC(C)(c1ccc(O)cc1)c1ccc(O)cc1] |
| | Diethyl- stibestrol | 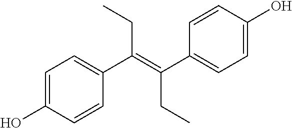 [SMILES: CC/C(=C(/CC)c1ccc(O)cc1)c1ccc(O)cc1] |
| Alkyl phenols | 4-tert amyl phenol | 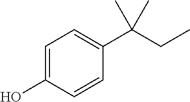 |
| Chloro phenol derivatives | Triclosan | 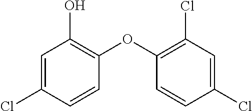 [SMILES: Oc1cc(Cl)ccc1Oc1ccc(Cl)cc1Cl] |
| Parabens | Propyl paraben | 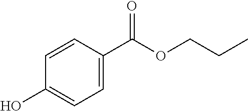 |
| Phtalates | Benzyl butyl phtalate | 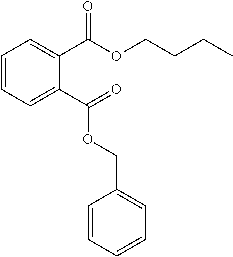 [SMILES: CCCCOC(=O)c1ccccc1C(=O)OCc1ccccc1] |
| | Dibutyl phtalate | 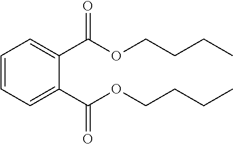 [SMILES: CCCCOC(=O)c1ccccc1C(=O)OCCCC] |
| | Di(2-éthyl exyl) phthalate (DEHP) | 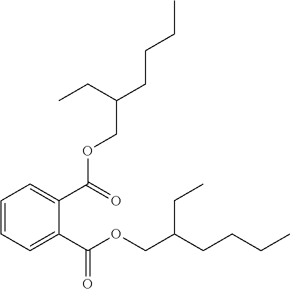 [SMILES: CCCCC(CC)COC(=O)c1ccccc1C(=O)OCC(CC)CCCC] |
| Camphor derivatives | 3- benylidene camphor | 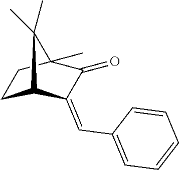 |

2.2.2. Determination of Subcytotoxic Concentrations

Known apoptotic agents were diluted in culture medium supplemented with 2.5% FBS and incubated for 24 hours. Before running the apoptosis assay, cell viability was determined using the Alamar blue assay to eliminate necrotic concentrations and only keep subcytotoxic concentrations of the agents. Alamar blue was diluted in culture medium to a working concentration of 9 μg/mL. The cells were incubated with the solution for 6 hours at 37° C. The fluorescence signal was read (λex=535 nm, λem=600 nm) using the Spark cytofluorometer.

2.2.3. Evaluation of Chromatin Condensation as a Hallmark of Apoptosis

The UV fluorescent probe Hoechst 33342 enters living and apoptotic cells, intercalating into DNA. The fluorescent signal is proportional to chromatin condensation in apoptosis. The cells were incubated with Hoechst 33342 at 10 μg/mL for 30 minutes at room temperature. The fluorescence signal was read (λex=360 nm, λem=460 nm) using a cytofluorometer (Spark).

3. Statistical Analysis

Means of at least three independent experiments were calculated and normalized to control. A one-way ANOVA followed by Dunnett's test were performed (α risk=5%) using GraphPad Prism 6 software (San Diego, CA, USA). Thresholds of significance were *p<0.05, **p<0.01, ***p<0.001, and ****p<0.0001 compared to control.

Results

JEG-3 Cells Behavior in Culture Medium Supplemented with Different Concentrations of Fetal Bovine Serum

Impact of Fetal Bovine Serum Concentration on Cell Proliferation

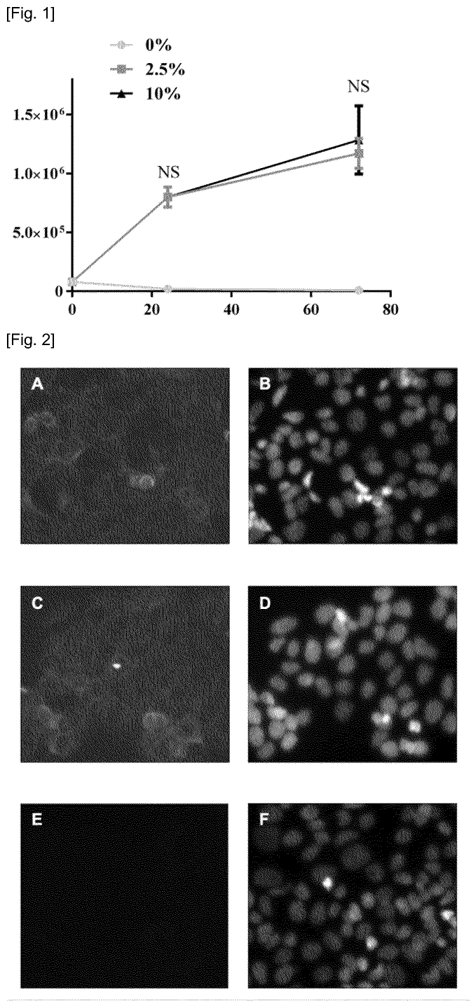

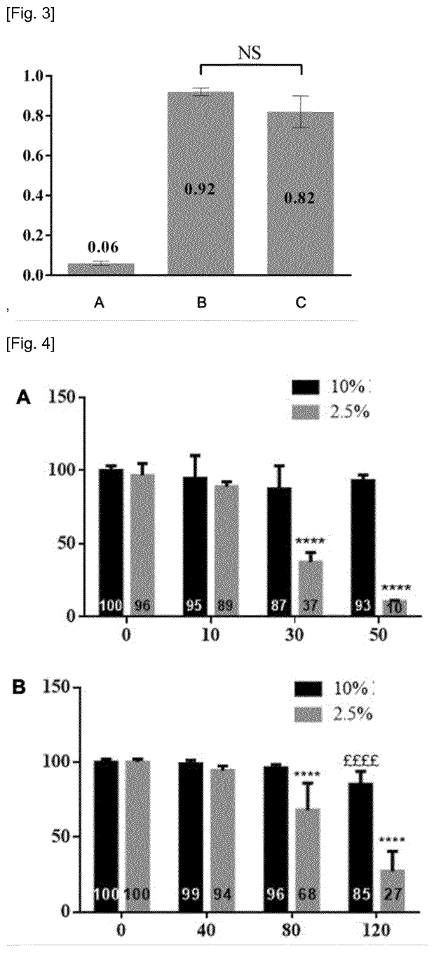

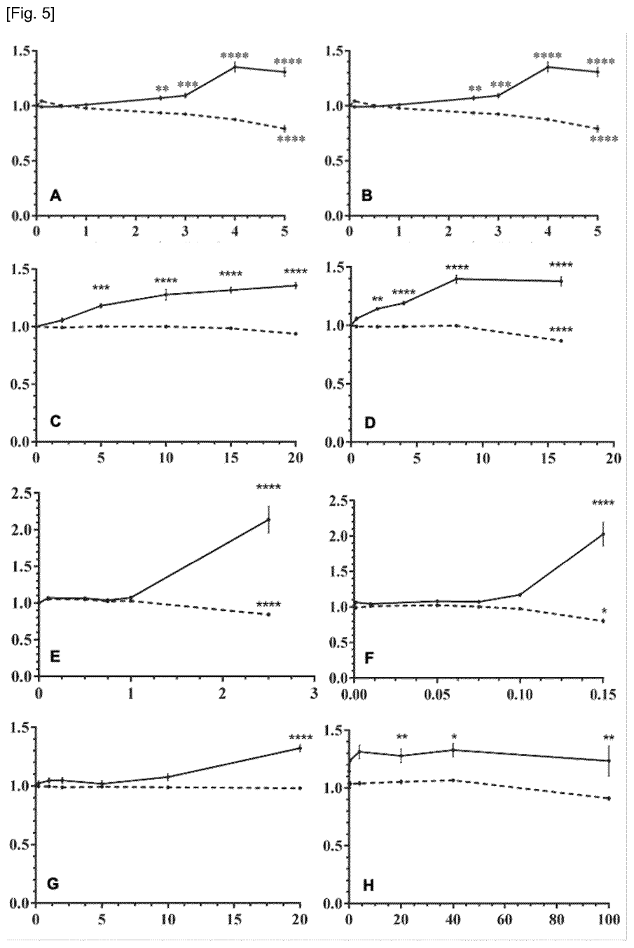

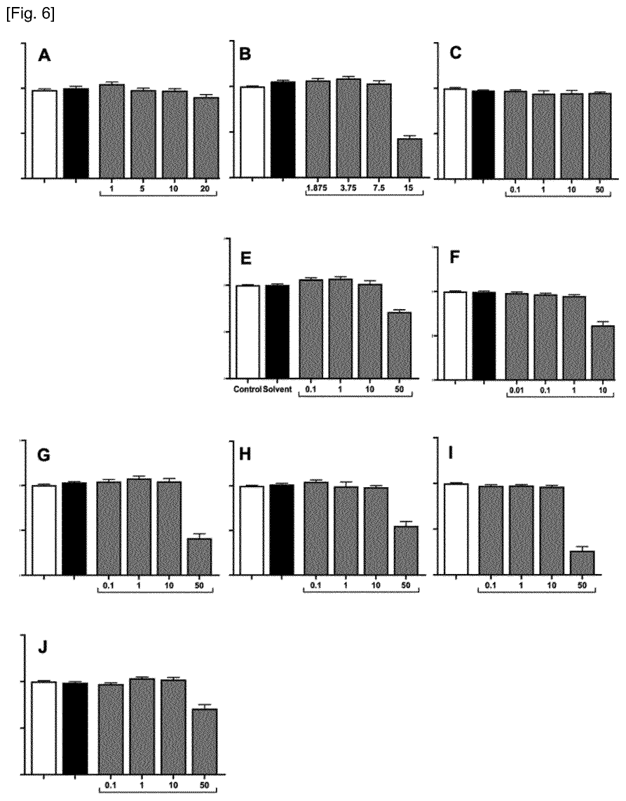

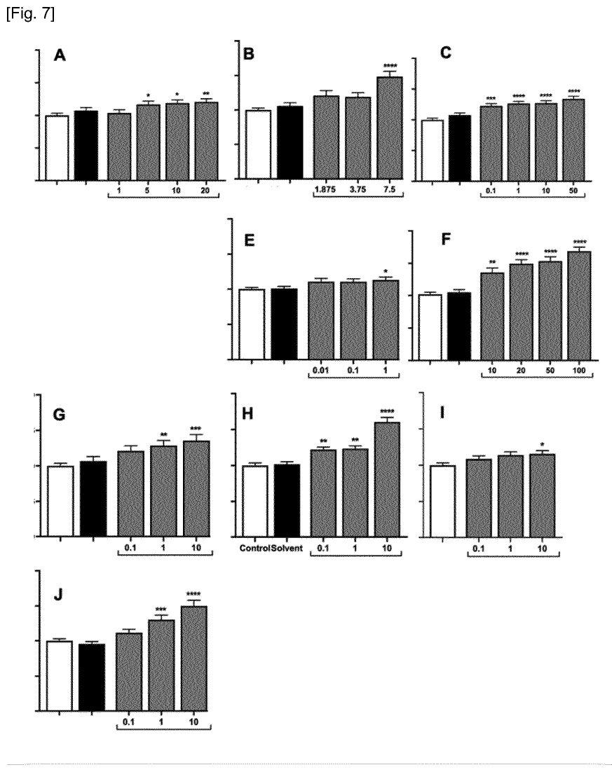

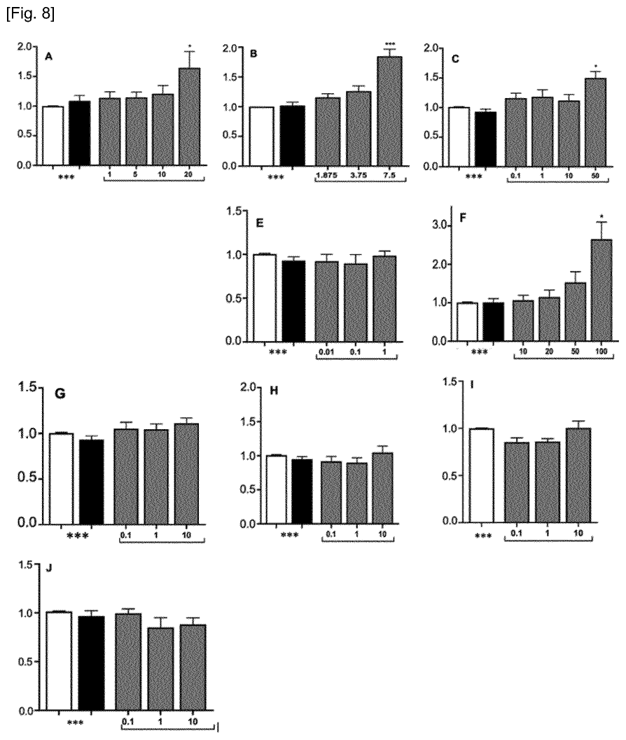

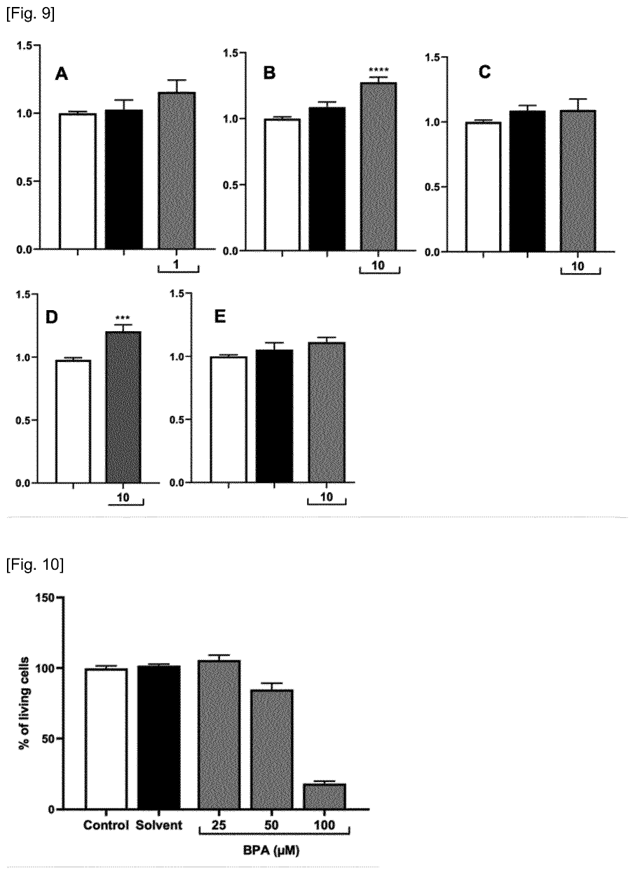

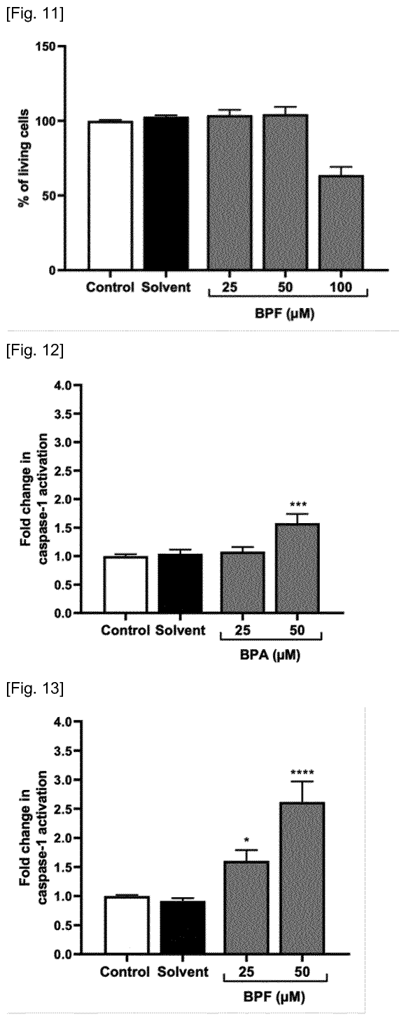

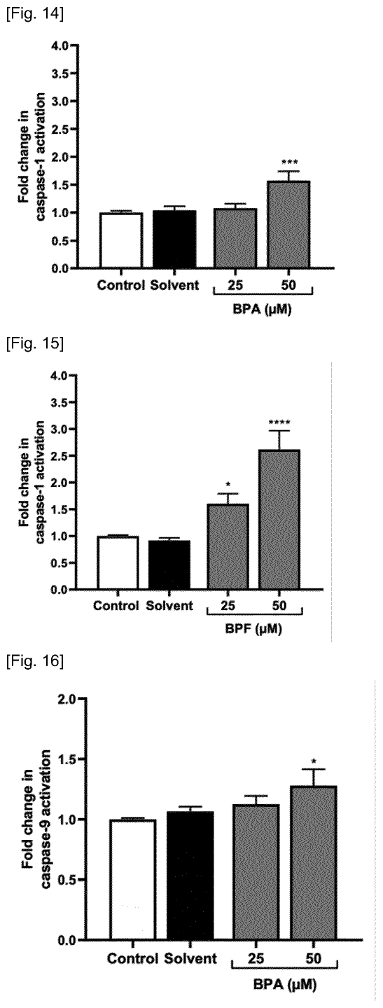

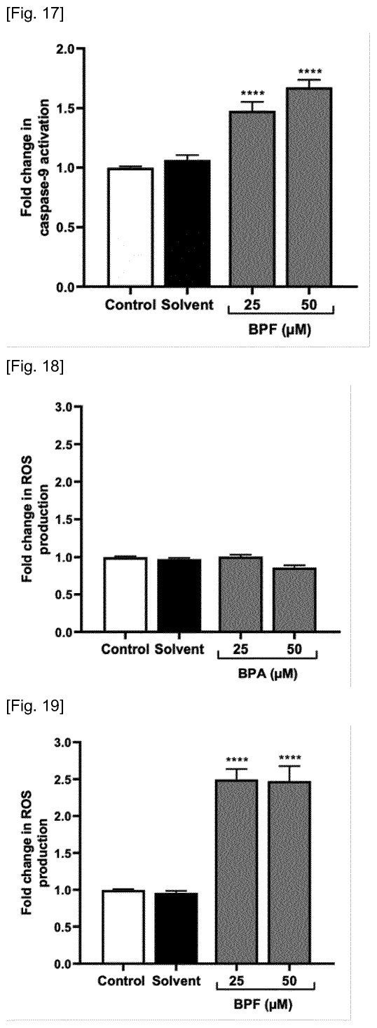

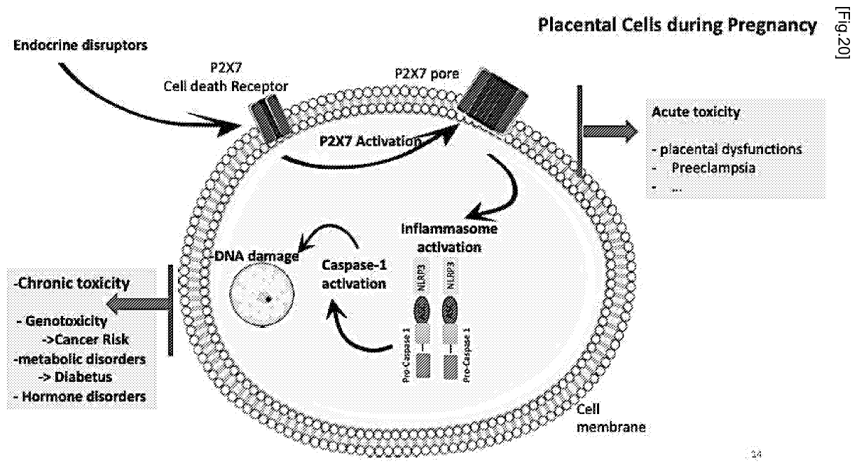

Three percentages of fetal bovine serum were used: 0%, 2.5% and 10% in culture medium (FIG. 1).

The percentage of living cells was dramatically decreased after 24 h in culture medium without FBS (0% FBS); as expected, JEG-3 cells were not able to proliferate without FBS due to a lack of nutritional and macromolecular factors. JEG-3 cell proliferation in culture medium supplemented with 2.5% was similar to proliferation in 10% FBS at 24 and 72 hours.

Cell Line Authentication by STR Analysis

The STR analysis was performed to compare nine STR core markers in JEG-3 cells in culture medium supplemented with 2.5% FBS to JEG-3 cells in 10% FBS (table 2).

TABLE 2
STR analysis of JEG-3 cells cultured in culture medium supplemented with
either 10% or 2.5% FBS. STR: Short Tandem Repeat; FBS: Fetal Bovine Serum

| Cell culture | Loci | | | | | | | | |
|---|---|---|---|---|---|---|---|---|---|
| conditions | D5S818 | D13S17 | D7S820 | D16S539 | vWA | THO1 | AMEL | TPOX | CSF1PO |
| 10% FBS | 10, 11 | 9, 11 | 10, 12 | 13, 14 | 16 | 9, 9.3 | X, Y | 8 | 11, 12 |
| 2.5% FBS | 10, 11 | 9, 11 | 10, 12 | 13, 14 | 16 | 9, 9.3 | X, Y | 8 | 11, 12 |

JEG-3 cells in 10% or 2.5% FBS expressed the same STR core markers. Reducing the percentage of FBS in culture medium of JEG-3 cells had no impact on DNA specific loci.

Expression of CK7 as a Marker of Placental Cells

CK7 is a well-known epithelial marker for trophoblast cells and is known to be expressed in JEG-3 cells cultured in 10% FBS. According to the inventors' microscopic observations, JEG-3 cells expressed similar levels of CK7 in 2.5% FBS and 10% FBS (FIGS. 2 and 3).

Quantification of Estradiol, Hyperglycosylated hCG and hPL Secretion by JEG-3 Cells

The inventors compared the secretion of placental hormones by JEG-3 cells in culture medium supplemented with 10% FBS to JEG-3 cells in 2.5% FBS. After 24 hours in either medium, the levels of each hormone were comparable (Table 3).

TABLE 3
Quantification of hormones in cell supernateants of JEG-3
cells in 10% or 2.5% FBS.

| | Estradiol (ng/mL) | hCG (mUl/mL) | hPL (μg/mL) |
|---|---|---|---|
| FBS 10% | 1 ± 0.2 | 1.9 ± 0.4 | 2.1 ± 0.6 |
| FBS 2.5% | 1 ± 0.6 | 2.2 ± 0.2 | 1.5 ± 0.3 |
| p value | >0.9999 (NS) | 0.7 (NS) | 0.7 (NS) |

Impact of Fetal Bovine Serum Concentration on SLS and PFOA Cytotoxicity

The inventors compared SLS and PFOA cytotoxicity in 2.5% FBS and 10% FBS, respectively (FIG. 4). At all tested concentrations, SLS diluted in culture medium supplemented with 10% FBS had no effect on JEG-3 cell viability. On the contrary, SLS diluted in culture medium supplemented with 2.5% FBS induced cytotoxicity at 30 μg/mL (37% of living cells, FIG. 4A) and 50 μg/mL (10% of living cells). PFOA cytotoxicity was observed at 80 μg/mL and 120 μg/mL in FBS 2.5% (68% and 27% of living cells, respectively, FIG. 4B) whereas only a slight loss of cell viability was observed at 120 μg/mL in FBS 10% (85% of viable cells). The classic concentration of FBS used for cell culture (10% of total volume) tends to mask SLS and PFOA cytotoxicity contrary to reduced FBS concentration (2.5%).

Based on the inventors' results, they pursue our study only using culture medium supplemented with FBS 2.5%; they renamed cells with these incubation conditions JEG-Tox.

Response of JEG-Tox Cells to Apoptosis Inducers

We studied chromatin condensation in JEG-Tox cells after incubation with apoptotic chemicals. Before assessing chromatin condensation, we selected subcytotoxic concentrations i.e. concentrations that result in % of living cells higher than 70 (data not shown). This threshold is recommended in ISO standards and OECD guidelines that assess cytotoxicity on monolayer cells. Subcytotoxic concentrations ranged from 0.1% to 5% for ethanol, from 0.03 to 150 μg/mL for quinalphos, from 2 to 20 μg/mL for bisphenol F, from 0.4 to 16 μg/mL for 4,4′DDT, from 0.1 to 2.5 μg/mL for BAC, from 0.0001 to 0.15% for phenoxyethanol, from 0.2 to 20 μg/mL for propylparaben and from 0.04 to 100 μg/mL for PFOA.

As shown in FIG. 5, all the apoptotic chemicals significantly induced chromatin condensation in JEG-Tox cells. Chromatin condensation was initiated with ethanol 2.5%, quinalphos 0.3 μg/mL, bisphenol F 5 μg/mL, 4,4′DDT 2 μg/mL, BAC 2.5 μg/mL, phenoxyethanol 0.15%, propylparaben 20 μg/mL and PFOA 20 μg/m; all those concentrations being in accordance with the literature in other cell types (25-31).

Discussion

Chemicals are more concentrated in the placenta than in maternal tissues. Exposure of pregnant women to hazardous chemicals and environmental pollutants like alcohol, pesticides, preservatives, or plasticizers can lead to decreased birth length and weight and increased infant mortality, alterations of developing nervous system and other vital organs, endocrine disruptions . . . .

Proteins present in FBS can bind chemicals thus masking their potential cytotoxicity and affecting cell response. It was previously proposed that the protein corona formed around particles greatly influences particle toxicity. High FBS concentrations used in growth medium (mainly 10%) are therefore not adapted for toxicity studies. Some of the inventors' previous studies on ocular and skin cell lines demonstrated that 2.5% FBS is a good compromise as serum total deprivation induces cell death (38-40). In this study, we compared placental JEG-3 cells behaviour in 2.5% FBS versus 10% FBS. The inventors first evaluated cell proliferation and observed that JEG-3 cells cultured in 2.5% or 10% FBS have similar proliferation rates, and as expected, cells in 0% FBS did not survive. The inventors second analysed STR core markers and concluded that JEG-3 cells had the same STR core markers and thus the same genotype whether they are cultured in 2.5% or in 10% FBS. The inventors third performed immunochemistry studies to ensure that JEG-3 cells in 2.5% FBS express cytokeratin 7 (CK7), a known marker of placental cells. The inventor's results showed that reducing the percentage of FBS in JEG-3 cells does not alter signatures of cell identity such as cell proliferation rate, DNA profile and specific protein expression. JEG-3 cells in 2.5% FBS released similar levels of hCG, hPL, and estradiol to JEG-3 cells in 10% FBS, and thus maintain the endocrine function of human placenta.

In the cytotoxicity study, the inventors did not observe any cell death when SLS was diluted in 10% FBS up to 50 μg/mL whereas when it was diluted in 2.5% FBS, SLS induced a dramatic loss of cell viability at 30 μg/mL. Cytotoxicity of PFOA was revealed at 200 μM when it was diluted in 2.5% FBS whereas only a slight loss of cell viability was observed at 300 μM when it was diluted in 10% FBS. It appears that JEG-3 cells in 2.5% FBS are more suitable for toxicological studies than JEG-3 cells in 10% FBS. The inventors renamed JEG-3 cells in 2.5% FBS JEG-Tox cells.

Apoptosis is suggested to be a key mechanism in placental dysfunction. A growing amount of data indeed suggests that uncontrolled placental apoptosis has side effects on both the placenta and maternal physiology. To validate JEG-Tox cells as a pertinent model for the evaluation of placental toxicity, the inventors checked whether they were able to trigger apoptosis after incubation with known apoptotic agents. We selected chemicals that pregnant women can be exposed to such as ethanol through alcohol consumption, preservatives present in cosmetics or drugs, pesticides and cookware coatings. In the inventor's experimental conditions, all the tested apoptotic chemicals induced chromatin condensation in JEG-Tox cells.

To conclude, reducing the percentage of FBS from 10%, which is the recommended concentration for cell growth, to 2.5% does not affect neither DNA profile, nor placental marker, nor hormone secretion, but reveals placental toxicity increasing cell sensitivity to chemicals contrary to FBS 10%. JEG-Tox cells can be of great value in placental toxicological studies, especially to study apoptosis that is at the origin of numerous severe pregnancy disorders.

### Example 2: Endocrine Disruptor Chemicals Activate P2X7 Receptor in Pregnancy Disorders

The inventors tested ten EDCs from different chemical families, three phthalates, two bisphenols, one camphor derivative, three phenols and one paraben, for their ability to activate P2X7 receptor and caspase-1 in human placental JEG-Tox cells, as defined in example 1. EDCS included here are member of chemicals families the most found in pregnant human and placenta; and they have been demonstrated to alter placental function and/or to induce pregnancy outcomes and complications.

Materials and Methods

Chemicals and Reagents

Cell culture reagents: Minimum essential Medium (MEM), Foetal Bovine Serum (FBS), 2 mM glutamine, 100 U/mL penicillin and 100 μg/mL streptomycin, trypsin-EDTA 0.05% and Phosphate Buffer Saline (PBS) were provided by Gibco (Paisley, UK) and cell culture material such as flasks and microplates by Corning (Schiphol-Rijk, The Netherlands). YO-PRO-1® probe was obtained from ThermoFisher Scientific (Waltham, Massachusetts, USA) and Caspase-Glo® 1 Inflammasome Assay from Promega (Madison, WI, USA). All chemicals were purchased from Sigma-Aldrich (Saint Quentin Fallavier, France).

Di(2-ethylhexyl)phtalate (DEHP) were dissolved in culture medium. Benzyl butyl phthalate, dibutyl phthalate and propylparaben were dissolved in absolute ethanol. Bisphenol A, diethylstilbestrol, 4-tert-amylphenol, triclosan and 3-benzylidene camphor were dissolved in DMSO. Stock solutions were stored at −20° C. and work solutions were obtained after a 1/1000 dilution in culture medium. The final concentration of absolute ethanol and DMSO on cells was less than or equal to 0.1%.

JEG-3 Cell Culture

The JEG-3 human trophoblast cell line, derived from a human placental carcinoma, was obtained from the American Type Culture Collection (ATCC HTB-36). Cells were cultured in minimum Essential medium, supplemented with 10% fetal bovine serum (FBS), 1% L-glutamine, 0.5% penicillin and streptomycinin, in 75 cm2 polystyrene flasks. Cell cultures were maintained in a cell culture incubator (37° C., saturated humidity, 5% CO₂). When the JEG-3 cells reached subconfluency, they were detached using trypsin-EDTA and counted. The cellular suspension was diluted and seeded either in 96-well microplates at a cellular density of 80,000 cells/mL or in 6-well microplates at 120,000 cells/mL, then kept at 37° C. for 24 h. The cells were incubated with EDCs according to example 1.

Cell Viability: Neutral Red Assay

Cell viability was evaluated using the Neutral Red assay. The Neutral Red solution at 0.4% (m/v in water) was diluted in cell culture medium to obtain a working concentration of 50 μg/mL. Neutral Red working solution was distributed in the plates for a 3-hour incubation time at 37° C. The cells were then rinsed with PBS and lysed with a solution of acetic acid-ethanol (ethanol 50.6%, water 48.4% and acetic acid 1%). After homogenization, the fluorescence signal was scanned (λexc=540 nm, λem=600 nm) with a Tecan Spark microplate reader (Mannedorf, Switzerland). The cell viability was calculated compared to the control cells (the fluorescence of the control cells as 100% viability).

P2X7 Receptor Activation: YO-PRO-1® Assay

P2X7 cell death receptor activation was evaluated using the YO-PRO-1® assay (Rat et al. J Biol Methods. 2017 Jan. 20; 4(1):e64). YO-PRO-1® probe only enters into cells after P2X7 receptor activation-induced pore opening, and binds to DNA, emitting fluorescence. A 1 mM YO-PRO-1 stock solution was diluted at 1/500 in PBS just before use and distributed into the wells of the microplate. After a 10-minute incubation time at room temperature, fluorescence signal was read (λex=491 nm, λem=509 nm) with a Spark cytofluorometer.

Caspase 1 Activity: Caspase-Glo® 1 Inflammasome Assay

Caspase 1 activity was evaluated using the Caspase-Glo® 1 1 Inflammasome Assay kit. The assay was performed according to the manufacturer's instructions. Luminescence was quantified with a Spark microplate reader.

Results Exploitation and Statistical Analysis

Results are expressed in percentage or fold change compared to control cells and presented as means of at least three independent experiments±standard errors of the mean.

Statistical analysis was performed using GraphPad Prism 8 software (GraphPad Software, La Jolla, CA). A one-way ANOVA followed by Dunnett's test with a risk set at 5% were performed. Thresholds of significance were ****p<0.0001, ***p<0.001, **p<0.01 and *p<0.05 compared to control cells.

Results

JEG-3 Cell Viability

The inventors investigated JEG-Tox cell viability after incubation with EDCs using the Neutral Red assay. Any concentration inducing a loss of cell viability greater than or equal to 30% was considered as cytotoxic (ISO, 2009).

No loss of cell viability was observed after 72 hours with neither bisphenol A up to 20 μM (FIG. 6), nor with 4-tert-amylphenol up to 50 μM (FIG. 6C). Propylparaben reduced slightly cell viability at 100 μM (87%, FIG. 6F).

Diethylstilbestrol at 15 μM and triclosan at 10 μM reduced cell viability to 40% and 60%, respectively (FIGS. 6B and 6E). At 50 μM 3-benzylidene camphor, dibutyl phthalate, benzyl butyl phthalate and DEHP significantly reduced cell viability to 70%, 70%, 55%, 40% and 25%, respectively (FIGS. 6D, 6J, 6H, 6G and 6I). Cytotoxic concentrations were excluded from subsequent assays.

P2X7 Receptor Activation

P2X7 pore opening, reflecting P2X7 receptor activation, was assessed using the fluorescent YO-PRO-1 assay. P2X7 receptor was significantly activated by all of the tested EDCs, except one.

Bisphenol A, DEHP and triclosan were the agents that induced the slightest fold changes (×1.21 at 20 μM in FIG. 7A, ×1.16 at 10 μM in FIG. 21 and ×1.13 at 1 μM in FIG. 7E, respectively compared to control).

Diethylstilbestrol, 4-tert-amylphenol, butyl benzyl phthalate and 3-benzylidene camphor inducted medium fold changes (×1.48 at 7.5 μM in FIG. 2B, ×1.36 at 50 μM in FIG. 7C, ×1.36 at 10 μM in FIG. 7G, 1.50 at 10 μM in FIG. 7J).

Propylparaben was the most potent activator (×1.69 at 100 μM, FIG. 7F), along with dibutyl phthalate (×1.6 at 10 μM, FIG. 7H).

Caspase-1 Activity

The bioluminescent Caspase-Glo® 1 assay was used to quantify caspase-1 activity.

Only the highest concentrations of bisphenol A, diethylstilbestrol, 4-tert-amylphenol, and propylparaben significantly activated caspase-1 compared to control (×1.64, ×1.84, ×1.5, ×5.1 and ×2.7, respectively compared to control, FIGS. 8A-C and 8F).

Triclosan, butyl benzyl phthalate, dibutyl phthalate, DEHP and 3-benzylidene camphor had no effect on caspase-1 activity (FIGS. 8E, 8G-J).

Caspase-9 Activity

P2X7 cell death receptor is also known to trigger the initiation of apoptosis, major cell death pathway. P2X7 receptor stimulates apoptosis that involves predominantly the calcium-dependent caspase-9-mediated mitochondrial pathway. We studied apoptosis through the assessment of the caspase-9 activity.

Benzyl butyl phthalate and DEHP at 10 μM were the chemical substances tested that induced significantly fold change in caspase-9 activity (×1.28 and ×1.20 at 10 μM, respectively compared to control, FIGS. 9B and 9D).

The following Table 4 summarize the results obtained in this example.

TABLE 4

| | | | | | P2X7 | Casp-1 |
|---|---|---|---|---|---|---|
| Family | Substance | formula | concentration | cytotoxicity | activation | activation |
| Bis-phenol | Bis-phenol A | 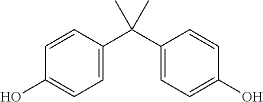 [SMILES: CC(C)(c1ccc(O)cc1)c1ccc(O)cc1] | 1-20 μM | No | 5-20 μM | 20 μM |
| | Diethyl- stibestrol | 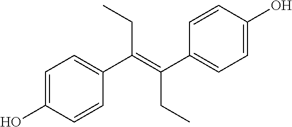 [SMILES: CC/C(=C(/CC)c1ccc(O)cc1)c1ccc(O)cc1] | 1.875-20 μM | 15 μM | 7.5 μM | 7.5 μM |
| Alkyl phenols | 4-tert amyl phenol | 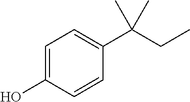 | 0.1-50 μM | no | 0.1-50 μM | 50 μM |
| Chloro phenol derivatives | Triclosan | 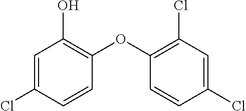 [SMILES: Oc1cc(Cl)ccc1Oc1ccc(Cl)cc1Cl] | 0.01-10 μM | 10 μM | 1 μM | No |
| Parabens | Propyl Paraben | 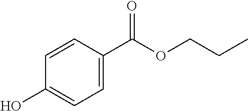 | 10-100 μM | no | 1-100 μM | 100 μM |
| Phtalates | Benzyl butyl phtalate | 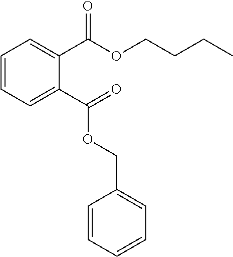 [SMILES: CCCCOC(=O)c1ccccc1C(=O)OCc1ccccc1] | 0.1-50 μM | 50 μM | 0.1-10 μM | no |
| | Dibutyl phtalate | 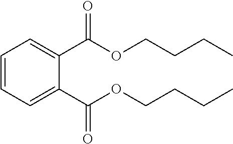 [SMILES: CCCCOC(=O)c1ccccc1C(=O)OCCCC] | 0.1-50 μM | 50 μM | 0.01-50 μM | no |
| | Di(2-éthyl exyl) phthalate (DEHP) | 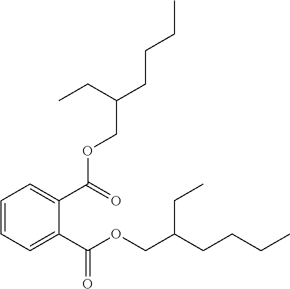 [SMILES: CCCCC(CC)COC(=O)c1ccccc1C(=O)OCC(CC)CCCC] | 0.1-50 μM | 50 μM | 10 μM | no |
| Camphor derivatives | 3- benylidene camphor | 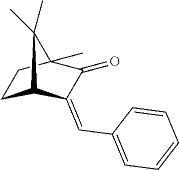 | 0.1-50 μM | 50 μM | 1-10 μM | no |

Discussion

Previous studies revealed that placenta should be considered as a fully-fledged target organ for toxic compounds and placental cell lines like JEG-Tox could represent useful tools for toxicological studies. P2X7 receptor is expressed by human placenta and JEG-Tox.

This study showed that the most of EDCs tested induce activation of P2X7 receptor in JEG-Tox. 9 of 10 EDCs tested have triggered P2X7 receptor activation: bisphenol A, diethylstilbestrol, 4-tert-amyl phenol, triclosan, propylparaben, butyl benzyl phthalate, dibutyl phthalate, DEHP and 3-benzylidene camphor. These EDCs belong to different chemical families from bisphenols to phthalates, through alkylphenols, parabens, and camphor derivatives.

Among these EDCs activating P2X7 receptor, 4 also activated caspase-1: bisphenol A, diethylstilbestrol, 4-tert-amylphenol and propylparaben. Caspase-1 is matured and activated via the formation of the inflammasome complex. Its activation can result in the production of activated inflammatory cytokines such as IL-1β and IL-18, but also cell death characterized by plasma-membrane permeability and release of proinflammatory intracellular compounds. It is an inflammasome-caspase-1-dependent programmed of cell death also known as pyroptosis. P2X7 receptor activation leads to cell death degeneration requiring caspase-1 activation.

This study brings out also that some EDCs tested activating P2X7 receptor have no effect on caspase-1 activity. This is the case of 5 EDCs: triclosan, butyl benzyl phthalate, dibutyl phthalate, DEHP and 3-benzylidene camphor. This can be explained by the fact that P2X7 receptors is also known to trigger cell degeneration by other pathways.

P2X7 cell death receptor is also known to trigger the initiation of apoptosis, major cell death pathway. P2X7 receptor stimulates apoptosis that involves predominantly the calcium-dependent caspase-9-mediated mitochondrial pathway. We studied apoptosis through the assessment of the caspase-9 activity.

Endocrine disruptor can induce DNA damage, genotoxic effects and cancer Risk via P2X7 activation.

As shown in FIG. 20, endocrine disruptors activate P2X7, itself activating inflammasome pathway that induce DNA damage through activation of Caspase-1.

A role in cancer cell growth and tumor progression has also been demonstrated.

The exposure of bisphenol A, diethylstilbestrol, 4-tert-amylphenol and propylparaben activate P2X7 receptor and caspase-1. Furthermore, these EDCs are found in placenta and their exposition during pregnancy are associated to placental dysfunctions and can lead to many adverse pregnancy outcomes. Additionally, it has been suggested that preterm birth and preeclampsia would be triggered by P2X7 receptor activation (Tsimis et al., 2017; Fodor et al., 2019). Our results suggest than EDCs can trigger placenta outcomes via the P2X7 receptor activation and inflammasome-caspase-1 activation.

The pro-inflammatory cytokines secreted following P2X7 activation include not only IL-1b and IL-18 but also IL-6 and IL-1a, albeit via an inflammasome independent route. Possibly one of the best characterized is the activation of the nuclear factor NF-jB, a transcription factor controlling expression of several inflammatory genes, including TNFa, COX-2 and IL-1b itself.

The seminal studies demonstrating P2X7R dependent activation of NF-κB in microglia, osteoclasts and osteoblasts were further confirmed by an increasing number of reports linking P2X7R pro-inflammatory activity to NF-κB nuclear translocation.

Conclusion

In this example, the inventors have found that endocrine disruptors induce alteration of the placenta by activating P2X7 degeneration receptors. The activation of the P2X7 receptor is therefore a biomarker to assess the deleterious effects of endocrine disruptors on the placenta. Indeed, its activation appears regardless of the chemical structure and class of molecules and whatever hormones will be disturbed later.

The human placental models are fully suited to the evaluation of endocrine disruptors. It is used to assess hormonal changes and to check for acute (P2X7 activation) or chronic adverse effects (genotoxicity, aromatase cyp19 disturbance and risk of reproductive disorders, metabolic disorders . . . ). It thus makes it possible to respond as best as possible to European definitions of endocrine disruptors and thus to their identification which must combine hormonal evaluation and deleterious effects.

This new generation of testing (i.e. hPLACENTOX-ED assays) that combines an innovative cellular model, hormonal measures, and new biomarkers of the deleterious effects of endocrine disruptors, make it possible to respond as best as possible to the new European regulations (chemicals—REACH, cosmetics, medical devices . . . ), which now require the assessment of the deleterious effects of endocrine disruptors and the identification of these effects with any new substances within the framework of these European regulations.

### Example 3: Bisphenol A and Bisphenol F Analysis

Over the last decade, human exposure to environmental endocrine disrupting chemicals and particularly to bisphenol A (BPA) seems to be a dominant threat for public health. BPA is a phenolic compound discovered in the late nineteenth century. It is used in a wide range of products like food containers and beverage, compact discs, personal protective equipment, sport equipment and medical equipment leading to multiple sources of exposure for the whole population.

BPA effects on placenta can thereby alter fetal programming. Placenta is indeed a crucial organ during pregnancy, acting as an endocrine organ and being an interface between the mother and fetus. Leclerc et al. showed that even very low concentrations of BPA are able to induce apoptosis, necrosis and inflammation of human trophoblastic cells in vitro.

BPA has been listed as a Substance of Very High Concern (SVHC) under REACh legislation, first because of its reprotoxic properties and then because of its endocrine disrupting properties. Its use has been limited and banned in baby bottles in Canada (2008), France (2010), and EU (2011). In France, since January 2015, BPA is forbidden in any food or beverage packaging. Such restrictions on BPA usage led manufactories to use alternative bisphenols such as bisphenol F. However, despite the increasing use of BPA structural analogs, there is limited information on potential placental and fetal toxicity of these molecules.

The inventors aim was to compare bisphenol A toxicity to its substitute, bisphenol F, on a human placental cell line in order to highlight the potential risks for placenta and then pregnancy.

1. Materials and Methods

Chemicals and Reagents

Bisphenol A is the compound of formula:

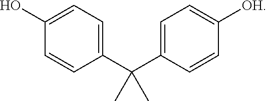 [SMILES: CC(C)(c1ccc(O)cc1)c1ccc(O)cc1]

Bisphenol F is the compound of formula:

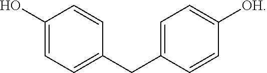 [SMILES: Oc1ccc(Cc2ccc(O)cc2)cc1]

Cell culture reagents: Minimum essential Medium (MEM), Fetal Bovine Serum (FBS), 2 mM glutamine, 100 U/mL penicillin and 100 μg/mL streptomycin, trypsin-EDTA 0.05% and Phosphate Buffer Saline (PBS) were provided by Gibco (Paisley, UK) and cell culture material such as flasks and microplates by Corning (Schiphol-Rijk, The Netherlands). YO-PRO-1® probe was obtained from ThermoFisher Scientific (Waltham, Massachusetts, USA) and Caspase-Glo® 1 Inflammasome Assay and Caspase-Glo® 9 Assay from Promega (Madison, WI, USA). All chemicals were purchased from Sigma-Aldrich (Saint Quentin Fallavier, France).

Bisphenol A and bisphenol F were dissolved in dimethylsulfoxyde (DMSO). Stock solutions were stored at −20° C. and work solutions were obtained after a 1/1000 dilution in culture medium. The final concentration of DMSO on cells was less than or equal to 0.1%.

JEG-3 Cell Culture

The JEG-3 human trophoblast cell line, derived from a human placental carcinoma, was obtained from the American Type Culture Collection (ATCC HTB-36). Cells were cultured in minimum Essential medium, supplemented with 10% fetal bovine serum (FBS), 1% L-glutamine, 0.5% penicillin and streptomycinin, in 75 cm² polystyrene flasks. Cell cultures were maintained in a cell culture incubator (37° C., saturated humidity, 5% CO2). When the JEG-3 cells reached subconfluency, they were detached using trypsin-EDTA and counted. The cellular suspension was diluted and seeded either in 96-well microplates at a cellular density of 80,000 cells/mL, in 24-well microplates at 160,000 cells/mL or in 6-well microplates at 120,000 cells/mL, then kept at 37° C. for 24 h. The cells were incubated with EDCs according to Olivier et al. who previously described the JEG-Tox model.

Cell Viability: Neutral Red Assay

Cell viability was evaluated using the Neutral Red assay. The Neutral Red solution at 0.4% (m/v in water) was diluted in cell culture medium to obtain a working concentration of 50 μg/mL. Neutral Red working solution was distributed in the plates for a 3-hour incubation time at 37° C. The cells were then rinsed with PBS and lysed with a solution of acetic acid-ethanol (ethanol 50.6%, water 48.4% and acetic acid 1%). After homogenization, the fluorescence signal was scanned (λexc=540 nm, λem=600 nm) with a Tecan Spark microplate reader (Mannedorf, Switzerland). The cell viability was calculated compared to the control cells (the fluorescence of the control cells as 100% viability).

P2X7 Receptor Activation: YO-PRO-1® Assay

P2X7 cell death receptor activation was evaluated using the YO-PRO-1® assay (Rat et al., 2017). YO-PRO-1® probe only enters into cells after P2X7 receptor activation-induced pore opening, and binds to DNA, emitting fluorescence. A 1 mM YO-PRO-1 stock solution was diluted at 1/500 in PBS just before use and distributed into the wells of the microplate. After a 10-minute incubation time at room temperature, fluorescence signal was read (λex=485 nm, λem=531 nm) with a Spark cytofluorometer.

Caspase 1 Activity: Caspase-Glo® 1 Inflammasome Assay

Caspase 1 activity was evaluated using the Caspase-Glo®1 Inflammasome Assay kit. The assay was performed according to the manufacturer's instructions. Luminescence was quantified with a Spark microplate reader.

Caspase 9 Activity: Caspase-Glo® Assay

Caspase 9 activity was evaluated using the Caspase-Glo® 9 Assay kit. The assay was performed according to the manufacturer's instructions. Luminescence was quantified with a Spark microplate reader.

Reactive Oxygen Species (ROS) Production: H2DCF-DA Assay

Intracellular ROS were measured using 2′,7′-dichlorodihydro-fluorescein diacetate (H2DCF-DA, Life Technologies), which is hydrolyzed by cell esterases in 2′,7′-dichlorodihydrofluorescein and oxidized by ROS in highly fluorescent 2′,7′-dichlorofluorescein. A 10 μM solution of H2DCF-DA was distributed into wells. After a 20-min incubation period at 37° C., the fluorescence signal was read (λex=485 nm, λem=535 nm) using a Spark microplate reader.

Cell Migration Assay:

JEG-Tox cells were seeded in a 24-wells microplate at 160 000 cells/mL for 24 h. Physical cell exclusion is created by placing an insert (Ibidi) on the culture surface before cell seeding. Inserts were removed (day 0) and cells were incubated with bisphenols for 24 h (day 1). The wound surface is analysed by the Image J software which allows quantification in arbitrary units. The ratio of the wound area observed on day 0 to that observed on day 1 corresponds to the cell migration factor.

Results Exploitation and Statistical Analysis

Results are expressed in percentage or fold change compared to control cells and presented as means of at least three independent experiments±standard errors of the mean.

Statistical analysis was performed using GraphPad Prism 8 software (GraphPad Software, La Jolla, CA). A one-way ANOVA followed by Dunnett's test with a risk set at 5% were performed. Thresholds of significance were ****p<0.0001, ***p<0.001, **p<0.01 and *p<0.05 compared to control cells.

2. Results

The inventors investigated JEG-Tox cell viability after incubation with EDCs using the Neutral Red assay. Any concentration inducing a loss of cell viability greater than or equal to 30% was considered as cytotoxic (ISO, 2009).

BPA and BPF at 100 μM reduced cell viability to 18% and 64% respectively (FIGS. 10 and 11).

P2X7 pore opening, reflecting P2X7 receptor activation, was assessed using the fluorescent YO-PRO-1 assay. P2X7 receptor was significantly activated by the two bisphenols tested. BPF was the most potent activator (×1.24 at 25 μM; ×1.46 at 50 μM compared to the control, FIG. 13). BPA inducted medium fold change (×1.26 at 25 μM and ×1.34 at 50 μM, FIG. 12).

The bioluminescent Caspase-Glo® 1 assay and Caspase-9 assay were used to quantify respectively caspase-1 and caspase-9 activity.

All concentration of BPF tested significantly activated caspase-1 compared to control (×1.60 and ×2.61 at 25 μM and 50 μM respectively, FIG. 15). Similar results are obtained for the caspase-9, 25 μM and 50 μM of BPF which significantly activated caspase-9 (×1.47 and ×1.67 respectively, FIG. 17) Only the highest concentration of BPA significantly activated caspase-1 (×1.57 at 50 μM, FIG. 14) and caspase-9 (×1.28, FIG. 16).

ROS production, reflecting oxidative stress, was assessed using the fluorescent H2DCF-DA assay. Only BPF induced oxidative stress but a significant ROS production (×2.5 at 25 μM and 50 μM, FIG. 18). In contrast, BPA had no effect on ROS production (FIG. 17).

### DISCUSSION AND CONCLUSION

In this example, the inventors have demonstrated that the cell culture according to the invention, i.e. JEG-3 cells cultured in a medium containing a low amount of serum (2.5%) is able to confirm that Bisphenol A is an endocrine disruptor according to the definition of the European Union, by measuring the activity of the P2X7 receptor. Moreover, the inventors have also identified that a compound having a structure very similar to Bisphenol A, namely Bisphenol F, corresponds also to an endocrine disruptor according to the definition of the European Union.

Therefore, Bisphenol A can be used in a kit for identifying if a compound is an endocrine disruptor as a positive control, the kit containing the JEG-3 cells cultured in law serum containing medium.

Caspase-1 and Caspase-9 activities, ROS production, DNA damages detection, and many other additional tests may be carried out in order to evaluate long term effect of a compound that was confirmed to be an endocrine disruptor.

The invention is not limited to the above description, and the skilled person can deduce additional embodiments of the invention.

1. A method for in vitro determining if a compound is an endocrine disruptor, said method comprising
a—contacting a compound liable to be an endocrine disruptor with a cell culture, the cell culture comprising a human endocrine placental cell cultured in a culture medium, the culture medium comprising minimal essential nutriments and a serum, wherein said serum represents from 1.5 to 3.5% weight compared to the total weight of the culture medium of the cell culture, then
b—measuring, in said culture medium contacted with a compound liable to be an endocrine disruptor, an expression level of a set of four hormones, the set comprising a first, a second, a third and a fourth hormone, each of the first, second, third and fourth hormone being secreted by the human endocrine placental cell, to obtain a measured expression level of the first, second, third and fourth hormones,
wherein the set comprises a progesterone hormone and a polypeptidic hormone or its derivatives,
c—comparing the measured expression level of each of the first, second, third and fourth hormone with a respective control expression level of each of the first, second, third and fourth hormone, the control expression level of each of the first, second, third and fourth hormone being measured in a culture medium from a cell culture containing a human endocrine placental cell which is not contacted with the compound liable to be an endocrine disruptor or which is contacted with a compound known not to be an endocrine disruptor,
d—measuring, in the human endocrine placental cell from the cell culture contacted with the compound liable to be an endocrine disruptor, an expression level and/or an activity, of a P2X7 membrane receptor protein, to obtain a measured expression level and/or activity of the P2X7 membrane receptor protein, and comparing the measured expression level and/or activity of the P2X7 membrane receptor protein with a control expression level and/or activity of the P2X7, said control expression level and/or activity of the P2X7 being measured in a human endocrine placental cell of a cell culture which is not contacted with the compound liable to be an endocrine disruptor or which is contacted with a compound known not to be an endocrine disruptor,
e—concluding that:
i—if the measured expression level of at least one of the first, second, third and fourth hormone is significatively different from the respective control of each of the first, second, third and fourth hormone, and the measured expression level and/or activity of the P2X7 membrane receptor protein is significatively different from its respective control, then the compound is an endocrine disruptor;
ii—if the measured expression level of at least one of the first, second, third and fourth hormone is significatively different from the respective control of each of the first, second, third and fourth hormone, but the measured expression level and/or activity of the P2X7 membrane receptor protein is not significatively different from its respective control, then it is not excluded that the compound is endocrine disruptor,
iii—if only the measured expression level of the measured expression level and/or activity of the P2X7 membrane receptor protein is significatively different from the respective control, then the compound is not an endocrine disruptor.

2. The method according to claim 1, the method further comprising:
measuring
the activation of inflammasome pathway, in the human endocrine placental cell from the cell culture contacted with the compound liable to be an endocrine disruptor to obtain a measured inflammasome activity; or
mitochondrial activity in the human endocrine placental cell from the cell culture contacted with the compound liable to be an endocrine disruptor, to obtain a measured mitochondria activity,
or both;
comparing
the measured inflammasome activity to a control activity of the inflammasome pathway, said control activity of the inflammasome pathway being measured in a human endocrine placental cell of a cell culture which is not contacted with the compound liable to be an endocrine disruptor or which is contacted with a compound known not to be an endocrine disruptor;
and/or
the measured mitochondria activity to a control activity of the mitochondria, said control activity of control activity of the mitochondria being measured in a human endocrine placental cell of a cell culture which is not contacted with the compound liable to be an endocrine disruptor or which is contacted with a compound known not to be an endocrine disruptor; and
d—concluding that,
if the measured inflammasome activity or the mitochondria activity is significantly different from the respective control inflammasome activity and the control mitochondria activity, then the compound is an endocrine disruptor, and
otherwise, it is not excluded that the compound is endocrine disruptor.

3. The method according to claim 1, wherein the method further comprises:
measuring the presence of DNA damages in said endocrine cells, to obtain a measured DNA fragmentation,
comparing the measured DNA damages to a control DNA damage, said control DNA damage being measured in a human endocrine placental cell of a cell culture which is not contacted with the compound liable to be an endocrine disruptor or which is contacted with a compound known not to be an endocrine disruptor, and
concluding that:
when it is not excluded that the compound is endocrine disruptor
if the measured DNA damages is significantly different from the control DNA fragmentation, then the compound is an endocrine disruptor having genotoxic effects, and
if the measured DNA damages is not significantly different from the control DNA damage, then it is not excluded that the compound is endocrine disruptor, and
when the compound is an endocrine disruptor,
if the measured DNA damages is significantly different from the control DNA fragmentation, then the compound is an endocrine disruptor having genotoxic effects, then the compound is an endocrine disruptor, and
if the measured DNA damages is not significantly different from the control DNA fragmentation, then the compound is an endocrine disruptor having no genotoxic effects.

4. The method according to claim 1, wherein the method further comprises:
measuring the expression in the culture medium of said endocrine cells of hormones induced upon carcinogenic stimulation, to obtain a measured carcinogenic stimulation,
comparing the measured carcinogenic stimulation to a control carcinogenic stimulation, said control carcinogenic stimulation being measured in a human endocrine placental cell of a cell culture which is not contacted with the compound liable to be an endocrine disruptor or which is contacted with a compound known not to be an endocrine disruptor, and
concluding that
when it is not excluded that the compound is endocrine disruptor
if the measured carcinogenic stimulation is significantly different from the control carcinogenic stimulation, then the compound is an endocrine disruptor having carcinogenic effects, and
if the measured carcinogenic stimulation is not significantly different from the control carcinogenic stimulation, then it is not excluded that the compound is endocrine disruptor, and
when the compound is an endocrine disruptor,
if the measured carcinogenic stimulation is significantly different from the control carcinogenic stimulation, then the compound is an endocrine disruptor having carcinogenic effects, and
if the measured carcinogenic stimulation is not significantly different from the control carcinogenic stimulation, then the compound is an endocrine disruptor having no carcinogenic effects.

5. The method according to claim 1, the method further comprising:
measuring the activity of the aromatase enzyme of said endocrine cells, to obtain a measured aromatase activity,
comparing the measured aromatase activity to a control aromatase activity, said control aromatase activity being measured in a human endocrine placental cell of a cell culture which is not contacted with the compound liable to be an endocrine disruptor or which is contacted with a compound known not to be an endocrine disruptor, and
d—concluding that
when it is not excluded that the compound is endocrine disruptor
if the measured aromatase activity is significantly different from the control aromatase activity, then the compound is an endocrine disruptor having effects on fertility, and
if the measured aromatase activity is not significantly different from the control aromatase activity, it is not excluded that the compound is endocrine disruptor, and
when the compound is an endocrine disruptor,
if the measured aromatase activity is significantly different from the control aromatase activity, then the compound is an endocrine disruptor having effects on fertility, and
if the measured aromatase activity is not significantly different from the control aromatase activity, then the compound is an endocrine disruptor having no effects on fertility.

6. The method according to claim 1, wherein there is a significant difference when the measured and the control values differ of +/−15%.

7. The method according to claim 1, wherein the peptidic hormones are beta Chorionic gonadotropin hormone or BhCG, or one of its derivative, such that a glycosyltated BhCG, and Human Placental Lactogen or hPL.

8. The method according to claim 1, wherein the activation of inflammasome pathway is measured by evaluating caspase-1 protein activity, and/or IL1β expression and/or secretion.

9. A kit comprising:
a cell culture comprising a human placental endocrine cell and a culture medium consisting of minimal essential nutriments and serum, wherein said serum represents from 1.5 to 3.5% weight, preferably about 2.5% weight compared to the total weight of the culture medium, and
means for measuring the expression of four hormones secreted by placental cells, the four hormones comprising a progesterone hormone and a polypeptidic hormone or its derivatives secreted by placental cells
and means for measuring the expression and/or activation of the P2X7 receptor.

10. A cell culture comprising:
a human endocrine placental cell; and
a culture medium consisting of minimal essential nutriments and serum,
wherein said serum represents from 1.5 to 3.5% weight, preferably about 2.5% weight compared to the total weight of the culture medium.

11. The cell culture according to claim 10, wherein said endocrine placental cell is a placental cell line.

12. The cell culture according to claim 10, wherein the endocrine placental cell is a cytotrophoblastic placental cell.

13. The cell culture according to claim 10, wherein the endocrine placental cell is strictly adherent to a support onto which the endocrine cells are cultured.

14. The cell culture according to claim 10, wherein said endocrine placental cell is the placental cell line JEG-3, in particular the placental cell line deposited at ATCC under the number ATCC HTB-36.

15. (canceled)

16. The method of claim 1, wherein the polypeptidic hormone is one of the group consisting of B Human chorionic gonadotrophin or BhCG, a glycosyltated form of BhCG, and Human Placental Lactogen or hPL.

17. The kit according to claim 9, wherein the polypeptidic hormone is one of the group consisting of B Human chorionic gonadotrophin or BhCG, a glycosyltated form of BhCG, and Human Placental Lactogen or hPL.

18. The cell culture according to claim 10, wherein said endocrine placental cell is a placental cell line deposited at ATCC under the number ATCC HTB-36.
